(19) United States
(12) Patent Application Publication
(10) Pub. No.: US 2022/0177580 A1
(43) Pub. Date: Jun. 9, 2022

(54) MODULATING ANTIBODY EFFECTOR FUNCTIONS

(71) Applicant: AMGEN INC., THOUSAND OAKS, CA (US)

(72) Inventors: SCOTT THOMAS KUHNS, Newbury Park, CA (US); RUPA PADAKI, Thousand Oaks, CA (US); QINGCHUN ZHANG, Oak Park, CA (US); WILLIAM S BRETZLAFF, Newbury Park, CA (US)

(73) Assignee: AMGEN INC., THOUSAND OAKS, CA (US)

(21) Appl. No.: 17/609,363

(22) PCT Filed: May 28, 2020
(86) PCT No.: PCT/US2020/035016

§ 371 Date: Nov. 5, 2021

Related U.S. Application Data
(60) Provisional application No. 62/843,919, filed on May 6, 2019.

(51) Int. Cl. C07K 16/28

(52) U.S. Cl. CPC C07K 16/283; C07K 2317/41; C07K 2317/732; C07K 2317/21

(57) ABSTRACT

Provided herein are methods of modulating Fc gamma Receptor (FcγR)-mediated cytotoxicity of an antibody composition. In exemplary embodiments, the method comprises (1) increasing or decreasing the amount of terminal β-galactose at the N-297 glycosylation site of panitumumab, or increasing or decreasing the amount of panitumumab molecules that comprise G1, G1a, G1b and/or G2 galactosylated glycan at the N-297 site, (2) increasing or decreasing the amount of panitumumab molecules that comprise fucosylated glycan at the N-297 site, or increasing or decreasing the amount of panitumumab molecules that comprise afucosylated glycan at the N-297 site, and (3) increasing or decreasing the amount of panitumumab molecules that comprise a high-mannose glycan at the N-297 site.

### FIELD OF THE INVENTION

The present invention relates generally to modulating effector functions of therapeutic antibodies.

### BACKGROUND

Monoclonal antibodies have become widely used as therapeutic agents for treatment of a wide range of metabolic, inflammatory and oncology disease states. The most common human antibody subclasses used as biotherapeutics, IgG1 and IgG2, have very different immunological properties and are usually selected for a drug candidate based on the desired mechanism of action. Target cell killing, as might be desirable for a cancer indication, would seek to take advantage of IgG1 mediated effector functions such as antibody-dependent cell-mediated cytotoxicity (ADCC), antibody dependent cellular phagocytosis (ADCP), and complement dependent cytotoxicity (CDC). While these can be easily mediated by IgG1 antibodies, IgG2's are traditionally thought to be incapable of bringing about such effects (Ravetch, J. V., and S. Bolland. 2001, Annu. Rev. Immunol. 19: 275-290). However, it was recently found that panitumumab, a human IgG2 EGFR antagonist indicated for the treatment of metastatic colorectal cancer, can mediate cytotoxicity (Schneider-Merck, J Immunol 2010; 184:512-520). This was shown to be mediated primarily through cells of the myeloid lineage (monocytes and neutrophils) and mediated by FcγRIIa, which stands in contrast to traditional ADCC, which is mediated by IgG1's through lymphoid-derived natural killer (NK) cells and associated with FcγRIIIa.

In the manufacturing of therapeutic monoclonal antibodies, ensuring the necessary product quality requires defining and monitoring key quality attributes that impact the product safety and efficacy. It has become well established that the specific glycan structures of IgG1 associated the conserved glycan in the Fc CH2 domain can strongly influence the interaction with the FcγRs that mediated ADCC, ADCP as well as C1q binding that initiates CDC (Reusch D, Tejada M L., Glycobiology 2015; 25:1325-34). However, there are no studies that investigate the influence of product quality attributes of an IgG2 molecule that influence immune mediated cytotoxic activity. Fc receptors are key immune regulatory receptors connecting the antibody mediated (Humoral) immune response to cellular effector functions. Fc gamma receptors on the surface of effector cells (like natural killer cells, macrophages or monocytes) bind to the Fc region of an IgG, which itself is bound to a target cell. Upon Fc-binding, a signaling pathway is triggered which results in the secretion of various substances that mediate the destruction of the targets cells. The level of cytotoxic effector function varies for human IgG subtypes. Human IgG1 and IgG3 bind better to FcγR's and thereby mediate higher effector functions as compared to IgG2 or IgG4 (Jefferis, R. 2007, Expert Opin. Biol. Ther. 7: 1401-1413; Daeron M, Fc receptor biology; Annu Rev Immunol. 1997; 15:203-234).

The contribution of IgG2-mediated cytotoxicity in therapeutic efficacy is not well understood, nor are the quality attributes that influence IgG2-mediated cytotoxicity. The quality attributes that are impactful and predictive of IgG2-mediated cytotoxicity, and therefore suitable to monitor during IgG2 antibody manufacturing, are not well established. Therefore, there is a need to understand how certain quality attributes influence IgG2-mediated cytotoxicity, and modulate such quality attributes accordingly.

### SUMMARY

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A method of modulating Fc gamma Receptor (FcγR)-mediated cytotoxicity of panitumumab, comprising increasing or decreasing the amount of terminal β-galactose at the N-297 glycosylation site of panitumumab, or increasing or decreasing the amount of panitumumab molecules that comprise G1, G1a, G1b, and/or G2 galactosylated glycan at the N-297 site.
E2. A method of increasing Fc gamma Receptor (FcγR)-mediated cytotoxicity of panitumumab, comprising increasing the amount of terminal β-galactose at the N-297 glycosylation site of panitumumab, or increasing the amount of panitumumab molecules that comprise G1, G1a, G1b, and/or G2 galactosylated glycan at the N-297 site.
E3. The method of E1 or E2, wherein an increase of about 1 percent of β-galactose increases FcγR-mediated cytotoxicity by about 0.55 percent to about 0.75 percent, such as about 0.55 percent, about 0.6 percent, about 0.65 percent, about 0.7 percent, or about 0.75 percent.
E4. A method of decreasing Fc gamma Receptor (FcγR)-mediated cytotoxicity of panitumumab, comprising decreasing the amount of terminal β-galactose at the N-297 glycosylation site of panitumumab, or decreasing the amount of panitumumab molecules that comprise G1, G1a, G1b, and/or G2 galactosylated glycan at the N-297 site.
E5. The method of E1 or E4, wherein a decrease of about 1 percent of β-galactose decreases FcγR-mediated cytotoxicity by about 0.55 percent to about 0.75 percent, such as about 0.55 percent, about 0.6 percent, about 0.65 percent, about 0.7 percent, or about 0.75 percent.
E6. The method of any one of E1-E5, wherein said FcγR is FcγRIIa.
E7. The method of any one of E1-E6, wherein said FcγR-mediated cytotoxicity is measured by an in vitro cytotoxicity assay, such as KILR™ Cytotoxicity Assay.
E8. The method of any one of E1-E7, wherein said FcγR-mediated cytotoxicity is FcγRIIa-mediated cellular cytotoxicity.
E9. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of terminal β-galactose at the N-297 glycosylation site of panitumumab, or increasing or decreasing the amount of panitumumab molecules that comprise G1, G1a, G1b, and/or G2 galactosylated glycan at the N-297 site; such that the difference in FcγR-mediated cytotoxicity between the panitumumab sample and the reference value is about 35% or less.
   E10. The method of E9, wherein the difference in FcγR-mediated cytotoxicity between the panitumumab sample and the reference value is about 30% or less, about 25% or less, about 20% or less, about 15% or less, about 10% or less, or about 5% or less.
   E11. The method of E9 or E10, wherein the FcγR-mediated cytotoxicity of the panitumumab sample is increased by increasing the amount of terminal β-galactose at the N-297 glycosylation site of panitumumab, or increasing the amount of panitumumab molecules that comprise G1, G1a, G1b, and/or G2 galactosylated glycan at the N-297 site.
   E12. The method of any one of E9-E11, wherein an increase of about 1 percent of β-galactose increases FcγR-mediated cytotoxicity by about 0.55 percent to about 0.75 percent, such as about 0.55 percent, about 0.6 percent, about 0.65 percent, about 0.7 percent, or about 0.75 percent.
   E13. The method of E9 or E10, wherein the FcγR-mediated cytotoxicity of the panitumumab sample is decreased by decreasing the amount of terminal β-galactose at the N-297 glycosylation site of panitumumab, or decreasing the amount of panitumumab molecules that comprise G1, G1a, G1b, and/or G2 galactosylated glycan at the N-297 site.
   E14. The method of any one of E9, E10, or E13, wherein a decrease of about 1 percent of β-galactose decreases FcγR-mediated cytotoxicity by about 0.55 percent to about 0.75 percent, such as about 0.55 percent, about 0.6 percent, about 0.65 percent, about 0.7 percent, or about 0.75 percent.
   E15. The method of any one of E9-E14, wherein said FcγR is FcγRIIa.
   E16. The method of any one of E9-E15, wherein said FcγR-mediated cytotoxicity is measured by an in vitro cytotoxicity assay, such as KILR™ Cytotoxicity Assay.
   E17. The method of any one of E9-E16, wherein said FcγR-mediated cytotoxicity is FcγRIIa-mediated cellular cytotoxicity.
   E18. A method of modulating Fc gamma Receptor (FcγR)-mediated cytotoxicity of panitumumab, comprising increasing or decreasing the amount of panitumumab molecules that comprise fucosylated glycan at the N-297 site.
   E19. A method of modulating Fc gamma Receptor (FcγR)-mediated cytotoxicity of panitumumab, comprising increasing or decreasing the amount of panitumumab molecules that comprise afucosylated glycan at the N-297 site.
   E20. A method of increasing Fc gamma Receptor (FcγR)-mediated cytotoxicity of panitumumab, comprising increasing the amount of panitumumab molecules that comprise fucosylated glycan at the N-297 site.
   E21. A method of increasing Fc gamma Receptor (FcγR)-mediated cytotoxicity of panitumumab, comprising decreasing the amount of panitumumab molecules that comprise afucosylated glycan at the N-297 site.
   E22. The method of any one of E18-E21, wherein an increase of about 1 percent of fucosylated panitumumab molecules increases FcγR mediated cytotoxicity by about 2.70 percent to about 3 percent, such as about 3.0 percent, about 2.95 percent, about 2.90 percent, about 2.85 percent, or about 2.70 percent.
   E23. The method of any one of E18-E21, wherein a decrease of about 1 percent of afucosylated panitumumab molecules increases FcγR mediated cytotoxicity by about 2.70 percent to about 3 percent, such as about 3.0 percent, about 2.95 percent, about 2.90 percent, about 2.85 percent, or about 2.70 percent.
   E24. A method of decreasing Fc gamma Receptor (FcγR)-mediated cytotoxicity of panitumumab, comprising decreasing the amount of panitumumab molecules that comprise fucosylated glycan at the N-297 site.
   E25. A method of decreasing Fc gamma Receptor (FcγR)-mediated cytotoxicity of panitumumab, comprising increasing the amount of panitumumab molecules that comprise afucosylated glycan at the N-297 site.
   E26. The method of any one of E18-E19 and E24-E25, wherein a decrease of about 1 percent of fucosylated panitumumab molecules decreases FcγR mediated cytotoxicity by about 2.70 percent to about 3 percent, such as about 3.0 percent, about 2.95 percent, about 2.90 percent, about 2.85 percent, or about 2.70 percent.
   E27. The method of any one of E18-E19 and E24-E25, wherein an increase of about 1 percent of afucosylated panitumumab molecules increases FcγR mediated cytotoxicity by about 2.70 percent to about 3 percent, such as about 3.0 percent, about 2.95 percent, about 2.90 percent, about 2.85 percent, or about 2.70 percent.
   E28. The method of any one of E18-E27, wherein said FcγR is FcγRIIa.
   E29. The method of any one of E18-E28, wherein said FcγR mediated cytotoxicity is measured by an in vitro cytotoxicity assay, such as KILR™ Cytotoxicity Assay.
   E30. The method of any one of E18-E29, wherein said FcγR mediated cytotoxicity is FcγRIIa-mediated cellular cytotoxicity.
   E31. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise fucosylated glycan at the N-297 site; such that the difference in FcγR-mediated cytotoxicity between the panitumumab sample and the reference value is about 35% or less.
   E32. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise afucosylated glycan at the N-297 site; such that the difference in FcγR-mediated cytotoxicity between the panitumumab sample and the reference value is about 35% or less.
   E33. The method of E31 or E32, wherein the difference in FcγR-mediated cytotoxicity between the panitumumab sample and the reference value is about 30% or less, about 25% or less, about 20% or less, about 15% or less, about 10% or less, or about 5% or less.
   E34. The method of any one of E31-E33, wherein the FcγR-mediated cytotoxicity of the panitumumab sample is increased by increasing the amount of panitumumab molecules that comprise fucosylated glycan at the N-297 site.
   E35. The method of any one of E31-E34, wherein an increase of about 1 percent of fucosylated panitumumab molecules increases FcγR mediated cytotoxicity by about 2.70 percent to about 3 percent, such as about 3.0 percent, about 2.95 percent, about 2.90 percent, about 2.85 percent, or about 2.70 percent.
   E36. The method of any one of E31-E33, wherein the FcγR-mediated cytotoxicity of the panitumumab sample is increased by decreasing the amount of panitumumab molecules that comprise afucosylated glycan at the N-297 site.
   E37. The method of any one of E31-E34 and E36, wherein a decrease of about 1 percent of afucosylated panitumumab molecules increases FcγR mediated cytotoxicity by about 2.70 percent to about 3 percent, such as about 3.0 percent, about 2.95 percent, about 2.90 percent, about 2.85 percent, or about 2.70 percent.
   E38. The method of any one of E31-E33, wherein the FcγR-mediated cytotoxicity of the panitumumab sample is decreased by decreasing the amount of panitumumab molecules that comprise fucosylated glycan at the N-297 site.
   E39. The method of any one of E31-E33 and E38, wherein a decrease of about 1 percent of fucosylated panitumumab molecules decreases FcγR mediated cytotoxicity by about 2.70 percent to about 3 percent, such as about 3.0 percent, about 2.95 percent, about 2.90 percent, about 2.85 percent, or about 2.70 percent.
   E40. The method of any one of E31-E33, wherein the FcγR-mediated cytotoxicity of the panitumumab sample is decreased by increasing the amount of panitumumab molecules that comprise afucosylated glycan at the N-297 site.
   E41. The method of any one of E31-E33 and E40, wherein an increase of about 1 percent of afucosylated panitumumab molecules decreases FcγR mediated cytotoxicity by about 2.70 percent to about 3 percent, such as about 3.0 percent, about 2.95 percent, about 2.90 percent, about 2.85 percent, or about 2.70 percent.
   E42. The method of any one of E31-E41, wherein said FcγR is FcγRIIa.
   E43. The method of any one of E31-E42, wherein said FcγR-mediated cytotoxicity is measured by an in vitro cytotoxicity assay, such as KILR™ Cytotoxicity Assay.
   E44. The method of any one of E31-E43, wherein said FcγR-mediated cytotoxicity is FcγRIIa-mediated cellular cytotoxicity.
   E45. A method of modulating Fc gamma Receptor (FcγR)-mediated cytotoxicity of panitumumab, comprising increasing or decreasing the amount of panitumumab molecules that comprise a high-mannose glycan at the N-297 site.
   E46. A method of increasing Fc gamma Receptor (FcγR)-mediated cytotoxicity of panitumumab, comprising decreasing the amount of panitumumab molecules that comprise a high-mannose glycan at the N-297 site.
   E47. The method of E45 or E46, wherein a decrease of about 1 percent of high-mannose glycan increases FcγR-mediated cytotoxicity by about 1.20 percent to about 1.40 percent, such as about 1.2 percent, about 1.25 percent, about 1.3 percent, about 1.35 percent, or about 1.40 percent.
   E48. A method of decreasing Fc gamma Receptor (FcγR)-mediated cytotoxicity of panitumumab, comprising increasing the amount of panitumumab molecules that comprise a high-mannose glycan at the N-297 site.
   E49. The method of E45 or E48, wherein a decrease of about 1 percent of high-mannose glycan decreases FcγR-mediated cytotoxicity by about 1.20 percent to about 1.40 percent, such as about 1.2 percent, about 1.25 percent, about 1.3 percent, about 1.35 percent, or about 1.40 percent.
   E50. The method of any one of E45-E49, wherein said high-mannose is Mannose-5 (Man-5).
   E51. The method of any one of E45-E50, wherein said FcγR is FcγRIIa.
   E52. The method of any one of E45-E51, wherein said FcγR-mediated cytotoxicity is measured by an in vitro cytotoxicity assay, such as KILR™ Cytotoxicity Assay.
   E53. The method of any one of E45-E52, wherein said FcγR-mediated cytotoxicity is FcγRIIa-mediated cellular cytotoxicity.
   E54. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise a high-mannose glycan at the N-297 site; such that the difference in FcγR-mediated cytotoxicity between the panitumumab sample and the reference value is about 35% or less.
   E55. The method of E54, wherein the difference in FcγR-mediated cytotoxicity between the panitumumab sample and the reference value is about 30% or less, about 25% or less, about 20% or less, about 15% or less, about 10% or less, or about 5% or less.
   E56. The method of E54 or E55, wherein the FcγR-mediated cytotoxicity of the panitumumab sample is increased by decreasing the amount of panitumumab molecules that comprise a high-mannose glycan at the N-297 site.
   E57. The method of any one of E54-E56, wherein a decrease of about 1 percent of high-mannose glycan increases FcγR-mediated cytotoxicity by about 1.2 percent to about 1.40 percent, such as about 1.2 percent, about 1.25 percent, about 1.3 percent, about 1.35 percent, or about 1.40 percent.
   E58. The method of E54 or E55, wherein the FcγR-mediated cytotoxicity of the panitumumab sample is decreased by increasing the amount of panitumumab molecules that comprise a high-mannose glycan at the N-297 site.
   E59. The method of any one of E54, E55, or E58, wherein an increase of about 1 percent of high-mannose glycan decreases FcγR-mediated cytotoxicity by about 1.2 percent to about 1.40 percent, such as about 1.2 percent, about 1.25 percent, about 1.3 percent, about 1.35 percent, or about 1.40 percent.
   E60. The method of any one of E54-E59, wherein said high-mannose is Mannose-5 (Man-5).
   E61. The method of any one of E54-E60, wherein said FcγR is FcγRIIa.
   E62. The method of any one of E54-E61, wherein said FcγR-mediated cytotoxicity is measured by an in vitro cytotoxicity assay, such as KILR™ Cytotoxicity Assay.
   E63. The method of any one of E54-E62, wherein said FcγR-mediated cytotoxicity is FcγRIIa-mediated cellular cytotoxicity.
   E64. A method of modulating Fc gamma Receptor (FcγR)-mediated cytotoxicity of panitumumab, comprising:
(i) increasing or decreasing the amount of terminal β-galactose at the N-297 glycosylation site of panitumumab, or increasing or decreasing the amount of panitumumab molecules that comprise G1, G1a, G1b, and/or G2 galactosylated glycan at the N-297 site;
(ii) increasing or decreasing the amount of panitumumab molecules that comprise fucosylated glycan at the N-297 site, or increasing or decreasing the amount of panitumumab molecules that comprise afucosylated glycan at the N-297 site; and/or
(iii) increasing or decreasing the amount of panitumumab molecules that comprise a high-mannose glycan at the N-297 site.
   E65. A method of increasing Fc gamma Receptor (FcγR)-mediated cytotoxicity of panitumumab, comprising:
(i) increasing the amount of terminal β-galactose at the N-297 glycosylation site of panitumumab, or increasing the amount of panitumumab molecules that comprise G1, G1a, G1b, and/or G2 galactosylated glycan at the N-297 site;
(ii) increasing the amount of panitumumab molecules that comprise fucosylated glycan at the N-297 site, or decreasing the amount of panitumumab molecules that comprise afucosylated glycan at the N-297 site; and/or
(iii) decreasing the amount of panitumumab molecules that comprise a high-mannose glycan at the N-297 site.
   E66. A method of decreasing Fc Receptor (FcγR)-mediated cytotoxicity of panitumumab, comprising:
(i) decreasing the amount of terminal β-galactose at the N-297 glycosylation site of panitumumab, or decreasing the amount of panitumumab molecules that comprise G1, G1a, G1b, and/or G2 galactosylated glycan at the N-297 site;
(ii) decreasing the amount of panitumumab molecules that comprise fucosylated glycan at the N-297 site, or increasing the amount of panitumumab molecules that comprise afucosylated glycan at the N-297 site; and/or
(iii) increasing the amount of panitumumab molecules that comprise a high-mannose glycan at the N-297 site.
   E67. An antibody composition produced by the method of any one of E1-E66.
   E68. A pharmaceutical composition comprising the antibody composition of E67 and a pharmaceutically acceptable carrier, diluent or excipient.

### BRIEF DESCRIPTION OF THE DRAWINGS

FIG. 1A is an illustration of the three types of N-glycans (oligomannose, complex and hybrid) and commonly used symbols for such saccharides. FIG. 1B is an illustration the major N-Linked glycans found in human IgGs at the N-glycosylation site asparagine (Asn) 297 with a representative attachment of an oligosaccharide structure. These glycans commonly comprises a core heptasaccharide and outer arms constructed by variable addition of fucose, N-acetylglucosamine (GlcNAc), galactose, sialic acid (SA), and bisecting N-GlcNAc. FIG. 1C is a summary of the structures of the three major glycan species evaluated in the cytotoxicity reporter gene assay, including afucosylated species (i.e. species lacking core-fucose, including G0 or G1), high mannose species (including M5 species) or terminal β-galactose species (i.e. terminal beta-galactose, including G1F or G2F) species. FIG. 1D provides a basic schematic flow chart summarizing glycan enrichment and engineered sample preparation. FIG. 1E is a diagram of the salvage pathway and the de novo pathway of fucose metabolism. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, GOLAC. See, e.g., Nordeen et al. 2000; Hirschberg et al. 2001.

FIG. 2A shows the FcγRIIa signaling activity as a function of β-galactosylation level. FIG. 2B shows representative dose-response curve overlay. A linear regression line (with the equations shown) was fit for a plot of the measured activity (FIG. 2A), with representative dose-response curves provided for the various activity levels within the correlated line graphs (FIG. 2B). This data demonstrated the quantitative nature and range of the ADCC reporter gene assay, and its suitability for the assessment of quality attributes that impact ADCC activity. Higher levels of β-galactose generally result in higher cytotoxicity.

FIG. 3A shows the FcγRIIa signaling activity as a function of afucosylation level. FIG. 3B shows representative dose-response curve overlay. A linear regression line (with the equations shown) was fit for a plot of the measured activity (FIG. 3A), with representative dose-response curves provided for the various activity levels within the correlated line graphs (FIG. 3B). Higher levels of afucosylation generally result in lower cytotoxicity.

FIG. 4A shows the FcγRIIa signaling activity as a function of high-mannose level. FIG. 4B shows representative dose-response curve overlay. A linear regression line (with the equations shown) was fit for a plot of the measured activity (FIG. 4A), with representative dose-response curves provided for the various activity levels within the correlated line graphs (FIG. 4B). Higher levels of high-mannose generally result in lower cytotoxicity.

FIG. 5 shows representative dose curve for PBMC mediated cell cytotoxicity with the enriched glycan samples using KILR assay.

FIGS. 6A-6D are graphs showing PBMC mediated cytotoxicity as a function of panitumumab afucosylation levels from donors with (A) HHVV, (B) HHFF, (C) RRFV, and (D) HHFV polymorphisms for FcγRIIa and FcγRIIIa receptors, respectively.

FIGS. 7A-7D are graphs showing PBMC mediated cytotoxicity as a function of panitumumab high mannose levels from donors with (A) HHVV, (B) HHFF, (C) RRFV, and (D) HHFV polymorphisms for FcγRIIa and FcγRIIIa receptors, respectively.

FIGS. 8A-8D are graphs showing PBMC mediated cytotoxicity as a function of panitumumab β-galactose levels from donors with (A) HHVF, (B) RRVF, (C) HHVV, and (D) RRFF polymorphisms for FcγRIIa and FcγRIIIa receptors, respectively.

FIG. 9A is a representative panitumumab dose curve for PBMCs cytotoxicity activity. FIG. 9B is a plot showing FcγR blocking using antibodies against indicated receptors or controls.

FIGS. 10A-10C are SPR sensograms showing panitumumab or controls binding to (A) FcγRI at up to 10 uM antibody; (B) FcγRIIIa-158F at up to 10 uM antibody; and (C) FcγRIIa-131H at up to 10 uM antibody.

FIGS. 11A-11B are SPR equilibrium binding curves for panitumumab in (A) fucose enriched and (B) afucose enriched samples bind to huFcgRIIa-131H with apparent K_{D}˜7.9 μM and 8 μM respectively.

### DETAILED DESCRIPTION

### 1. Overview

The most common human antibody subclasses used as biotherapeutics, IgG1 and IgG2, have very different immunological properties. Common effector functions, such as antibody-dependent cell-mediated cytotoxicity (ADCC), can be an important mechanism of action for IgG1 antibodies. Previously, IgG2 antibodies were not known to exhibit effector functions. However, recently, it has been shown that panitumumab can mediate cytotoxic effect similar to ADCC by engaging FcγRIIa. This is in contrast to conventional ADCC mediated by IgG1 engaging with FcγRIIIa.

Panitumumab is a human IgG2 monoclonal antibody that binds to human epidermal growth factor receptor (also known as EGF receptor, EGFR, ErbB-1 and HER1). Panitumumab has an approximate molecular weight of 147 kD. The heavy chain and light chain sequences are shown in Table 1 as SEQ ID Nos. 1 and 2, respectively. Panitumumab has two N-glycosylation sites located in the 2nd constant domain of each heavy chain. The N-glycosylation site is commonly referred to as residue N-297 according to the Kabat EU numbering. The actual residue number is residue 295 of SEQ ID NO:1.

As described and exemplified herein, the inventors conducted an extensive study into mechanisms by which cytotoxicity of panitumumab is mediated, and product quality attributes that affect the FcγR-mediated cytotoxicity levels of panitumumab. The inventors discovered that cytotoxicity was primarily mediated by the myeloid lineage cells (monocytes, macrophages and neutrophils). The inventors then investigated the impact of the different glycans in this IgG2 molecule on effector functions. Using sensitive cytotoxicity assays in combination with glycoengineered forms of panitumumab, the inventor discovered that the impact of the different glycans on FcγRIIa-mediated cell killing can be substantial and variable depending on the glycoforms.

For example, galactosylation at the N-297 site showed a positive correlation in the reporter genes, while the afucosylation levels and high-mannose levels at the N-297 site showed an inverse correlation to cell killing. Therefore, the FcγR-mediated cytotoxicity of panitumumab can be increased by (1) increasing the galatosylation level at the N-297 site; (2) decreasing the afucosylation level at the N-297 site; and/or (3) decreasing the high-mannose level at the N-297 site. Conversely, the FcγR-mediated cytotoxicity of panitumumab can be decreased by (1) decreasing the galatosylation level at the N-297 site; (2) increasing the afucosylation level at the N-297 site; and/or (3) increasing the high-mannose level at the N-297 site.

Panitumumab is currently produced in genetically engineered mammalian (Chinese hamster ovary) cells. During recombination production process, glycan moieties are attached to the antibody through post-translational modification. The discoveries made by the inventors herein provide a quantifiable relationship between glycoform profiles of panitumumab and its cytotoxicity. The discovery can be used to modulate the glycosylation pattern during the CHO-cell production process, such that the cytotoxicity level meets a desired reference level.

### 2. Definitions

“Panitumumab” (trade names Vectibix®) refers to a human monoclonal antibody comprising a heavy chain comprising SEQ ID NO:1, and a light chain comprising SEQ ID NO:2. The amino acid sequences of the heavy and light chains of denosumab is shown in Table 1. Nucleic acid sequences encoding SEQ ID Nos: 1 and 2 are shown as SEQ ID Nos. 3 and 4, respectively. As illustrated in the examples, glycan profiles of panitumumab may vary.

TABLE 1
Sequences of panitumumab

| | Sequence |
|---|---|
| Heavy chain amino | QVQLQESGPG LVKPSETLSL TCTVSGGSVS SGDYYWTWIR QSPGKGLEWI |
| acid sequence | GHIYYSGNTN YNPSLKSRLT ISIDTSKTQF SLKLSSVTAA DTAIYYCVRD |
| (SEQ ID NO: 1) | RVTGAFDIWG QGTMVTVSSA STKGPSVFPL APCSRSTSES TAALGCLVKD |
| N-297 | YFPEPVTVSW NSGALTSGVH TFPAVLQSSG LYSLSSVVTV PSSNFGTQTY |
| glycosylation | TCNVDHKPSN TKVDKTVERK CCVECPPCPA PPVAGPSVFL FPPKPKDTLM |
| site is shown | 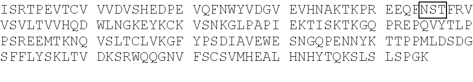 |
| Light chain | DIQMTQSPSS LSASVGDRVT ITCQASQDIS NYLNWYQQKP GKAPKLLIYD |
| amino acid | ASNLETGVPS RFSGSGSGTD FTFTISSLQP EDIATYFCQH FDHLPLAFGG |
| sequence | GTKVEIKRTV AAPSVFIFPP SDEQLKSGTA SVVCLLNNFY PREAKVQWKV |
| (SEQ ID NO: 2) | DNALQSGNSQ ESVTEQDSKD STYSLSSTLT LSKADYEKHK VYACEVTHQG |
| | LSSPVTKSFN RGEC |
| Heavy chain | 1 40 |
| nucleotide | (1) ATGGACCTCCTGTGCAAGAACATGAAACACCTGTGGTTCT |
| sequence | 41 80 |
| (SEQ ID NO: 3) | (41) TCCTCCTCCTGGTGGCAGCTCCCAGATGGGTCCTGTCCCA |
| | 81 120 |
| | (81) GGTACAGCTGCAGGAGTCGGGCCCAGGACTGGTGAAGCCT |
| | 121 160 |
| | 121) TCGGAGACCCTGTCCCTCACCTGCACTGTCTCTGGTGGCT |
| | 161 200 |
| | 161) CCGTCAGCAGTGGTGATTACTACTGGACCTGGATCCGGCA |
| | 201 240 |
| | 201) GTCCCCAGGGAAGGGACTGGAGTGGATTGGACACATCTAT |
| | 241 280 |
| | 241) TACAGTGGGAACACCAATTATAACCCCTCCCTCAAGAGTC |
| | 281 320 |
| | 281) GACTCACCATATCAATTGACACGTCCAAGACTCAGTTCTC |
| | 321 360 |
| | 321) CCTGAAGCTGAGTTCTGTGACCGCTGCGGACACGGCCATT |
| | 361 400 |
| | 361) TATTACTGTGTGCGAGATCGAGTGACTGGTGCTTTTGATA |
| | 401 440 |
| | 401) TCTGGGGCCAAGGGACAATGGTCACCGTCTCTTCAGCTAG |
| | 441 480 |
| | 441) CACCAAGGGCCCATCGGTCTTCCCCCTGGCGCCCTGCTCC |
| | 481 520 |
| | 481) AGGAGCACCTCCGAGAGCACAGCGGCCCTGGGCTGCCTGG |
| | 521 560 |
| | 521) TCAAGGACTACTTCCCCGAACCGGTGACGGTGTCGTGGAA |
| | 561 600 |
| | 561) CTCAGGCGCTCTGACCAGCGGCGTGCACACCTTCCCAGCT |
| | 601 640 |
| | 601) GTCCTACAGTCCTCAGGACTCTACTCCCTCAGCAGCGTGG |
| | 641 680 |
| | 641) TGACCGTGCCCTCCAGCAACTTCGGCACCCAGACCTACAC |
| | 681 720 |
| | 681) CTGCAACGTAGATCACAAGCCCAGCAACACCAAGGTGGAC |
| | 721 760 |
| | 721) AAGACAGTTGAGCGCAAATGTTGTGTCGAGTGCCCACCGT |
| | 761 800 |
| | 761) GCCCAGCACCACCTGTGGCAGGACCGTCAGTCTTCCTCTT |
| | 801 840 |
| | 801) CCCCCCAAAACCCAAGGACACCCTCATGATCTCCCGGACC |
| | 841 880 |
| | 841) CCTGAGGTCACGTGCGTGGTGGTGGACGTGAGCCACGAAG |
| | 881 920 |
| | 881) ACCCCGAGGTCCAGTTCAACTGGTACGTGGACGGCGTGGA |
| | 921 960 |
| | 921) GGTGCATAATGCCAAGACAAAGCCACGGGAGGAGCAGTTC |
| | 961 1000 |
| | 961) AACAGCACGTTCCGTGTGGTCAGCGTCCTCACCGTTGTGC |
| | 1001 1040 |
| | 1001) ACCAGGACTGGCTGAACGGCAAGGAGTACAAGTGCAAGGT |
| | 1041 1080 |
| | 1041) CTCCAACAAAGGCCTCCCAGCCCCCATCGAGAAAACCATC |
| | 1081 1120 |
| | 1081) TCCAAAACCAAAGGGCAGCCCCGAGAACCACAGGTGTACA |
| | 1121 1160 |
| | 1121) CCCTGCCCCCATCCCGGGAGGAGATGACCAAGAACCAGGT |
| | 1161 1200 |
| | 1161) CAGCCTGACCTGCCTGGTCAAAGGCTTCTACCCCAGCGAC |
| | 1201 1240 |
| | 1201) ATCGCCGTGGAGTGGGAGAGCAATGGGCAGCCGGAGAACA |
| | 1241 1280 |
| | 1241) ACTACAAGACCACACCTCCCATGCTGGACTCCGACGGCTC |
| | 1281 1320 |
| | 1281) CTTCTTCCTCTACAGCAAGCTCACCGTGGACAAGAGCAGG |
| | 1321 1360 |
| | 1321) TGGCAGCAGGGGAACGTCTTCTCATGCTCCGTGATGCATG |
| | 1361 1400 |
| | 1361) AGGCTCTGCACAACCACTACACGCAGAAGAGCCTCTCCCT |
| | 1401 1416 |
| | 1401) GTCTCCGGGTAAATGA |
| Light chain | 1 40 |
| nucleotide | 1) ATGGACATGAGGGTCCCTGCTCAGCTCCTGGGGCTCCTGC |
| sequence | 41 80 |
| (SEQ ID NO: 4) | 41) TGCTCTGGCTCTCAGGTGCCAGATGTGACATCCAGATGAC |
| | 81 120 |
| | 81) CCAGTCTCCATCCTCCCTGTCTGCATCTGTAGGAGACAGA |
| | 121 160 |
| | 121) GTCACCATCACTTGCCAGGCGAGTCAGGACATCAGCAACT |
| | 161 200 |
| | 161) ATTTAAATTGGTATCAGCAGAAACCAGGGAAAGCCCCTAA |
| | 201 240 |
| | 201) ACTCCTGATCTACGATGCATCCAATTTGGAAACAGGGGTC |
| | 241 280 |
| | 241) CCATCAAGGTTCAGTGGAAGTGGATCTGGGACAGATTTTA |
| | 281 320 |
| | 281) CTTTCACCATCAGCAGCCTGCAGCCTGAAGATATTGCAAC |
| | 321 360 |
| | 321) ATATTTCTGTCAACACTTTGATCATCTCCCGCTCGCTTTC |
| | 361 400 |
| | 361) GGCGGAGGGACCAAGGTGGAGATCAAACGAACTGTGGCTG |
| | 401 440 |
| | 401) CACCATCTGTCTTCATCTTCCCGCCATCTGATGAGCAGTT |
| | 441 480 |
| | 441) GAAATCTGGAACTGCCTCTGTTGTGTGCCTGCTGAATAAC |
| | 481 520 |
| | 481) TTCTATCCCAGAGAGGCCAAAGTACAGTGGAAGGTGGATA |
| | 521 560 |
| | 521) ACGCCCTCCAATCGGGTAACTCCCAGGAGAGTGTCACAGA |
| | 561 600 |
| | 561) GCAGGACAGCAAGGACAGCACCTACAGCCTCAGCAGCACC |
| | 601 640 |
| | 601) CTGACGCTGAGCAAAGCAGACTACGAGAAACACAAAGTCT |
| | 641 680 |
| | 641) ACGCCTGCGAAGTCACCCATCAGGGCCTGAGCTCGCCCGT |
| | 681 711 |
| | 681) CACAAAGAGCTTCAACAGGGGAGAGTGTTAG |

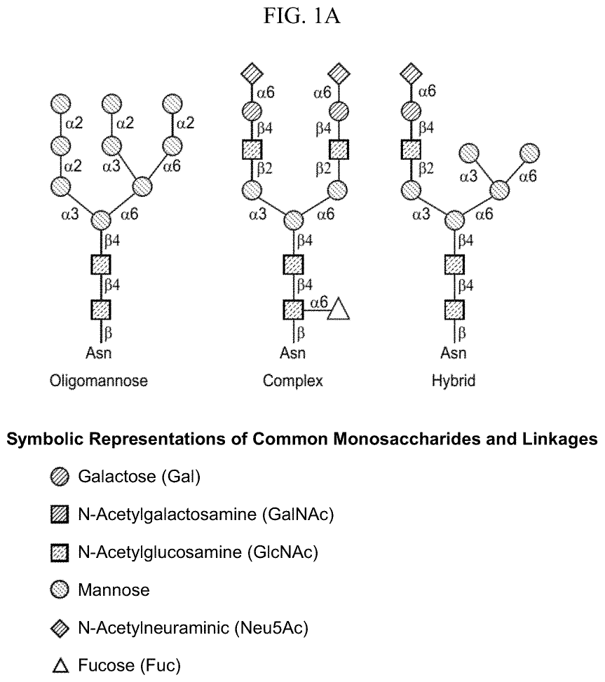

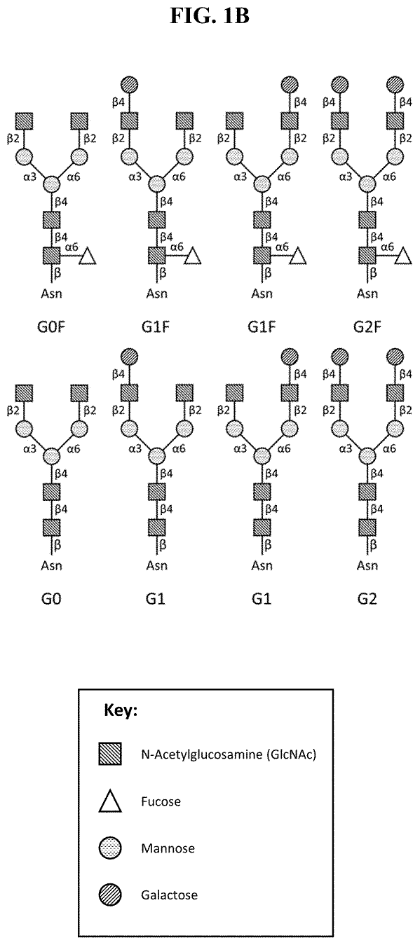

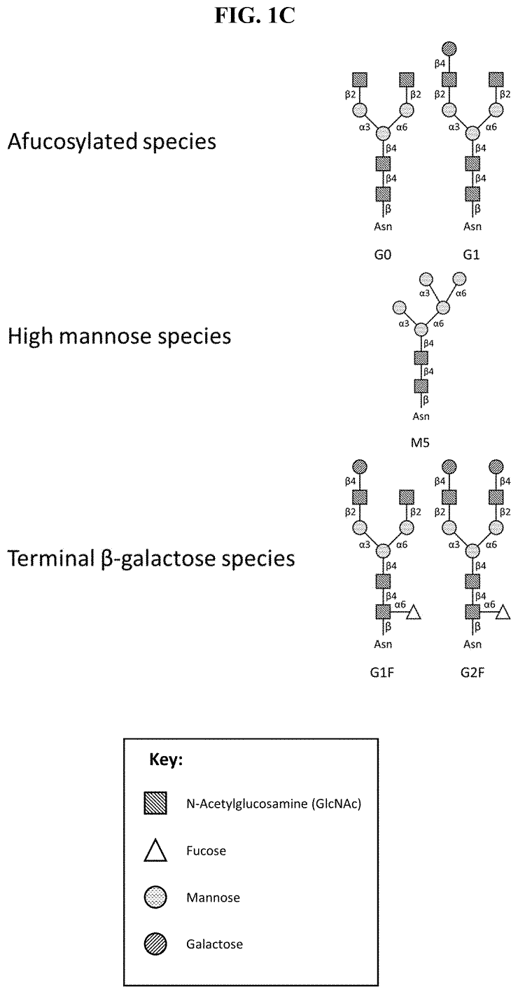

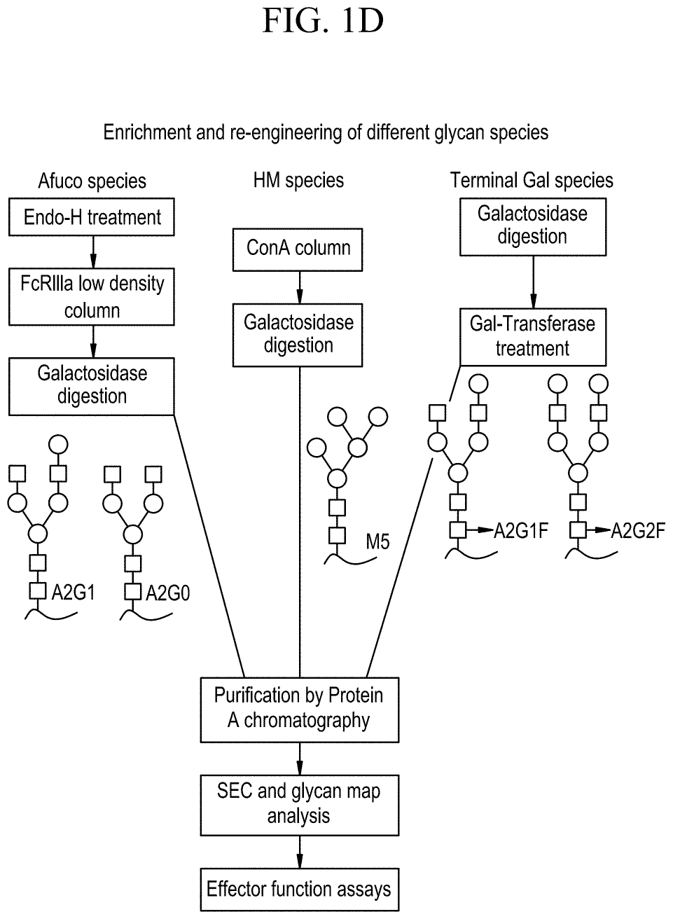

The term “glycan”, “glycans”, “glycoform” or “glycoforms” refers to oligomers of monosaccharide species that are connected by various glycosidic bonds. Examples of monosaccharides commonly found in mammalian N-linked glycans include hexose (Hex), glucose (Glc), galactose (Gal), mannose (Man) and N-acetylglucosamine (GlcNAc). The major N-glycan species found on recombinant IgG2 antibodies include fucose, galactose, mannose, sialic acid and GlcNAc, as depicted in FIGS. 1B, 1C, and Table 2. In case of panitumumab, the glycan oligosaccharide structures are linked to the N-glycosylation site at Asn-297 (Kabat EU numbering), and are generally composed of a core heptasaccharide with outer arms constructed by variable addition of fucose, N-acetylglucosamine (GlcNAc), galactose, sialic acid (SA), and bisecting N-GlcNAc. Each of the potential oligosaccharide structures may be abbreviated as follows: G0, G1, or G2 referring to the core GlcNAc and mannose oligosaccharide structure having zero, one or two terminal galactose molecules, respectively. Within G1, two additional structures, abbreviated G1a and G1b, may be present with G1a or G1b referring to whether the terminal galactose group is attached to either the 6-arm or the 3-arm of the core structure. See FIGS. 1B and 1C. When fucosylated (i.e. a fucose group is attached to the core glycan structure), the G0, G1 (G1a/G1b), or G2 forms may be abbreviated G0F, G1F (G1aF/G1bF), or G2F. When sialic acid is present, these abbreviations contain a “S” such that, for example, G2FS2 refers to a glycan having two galactose, a fucose and two sialic acid groups. Additional glycans linked to antibodies may also exist including high mannose (HM) structures, which are formed by the incorporation of additional mannose groups, including the high mannose species (e.g., “Man 5” or “M5” as shown in FIG. 1C and Table 2). As used herein, the term “glycan” or “glycans” refers to any of the oligomers of monosaccharide species described herein or any other oligomers of monosaccharaide species linked to an antibody.

The N-glycosylation sites of an IgG2 (located at the 2nd constant domain of the heavy chain) is typically referred to as N-297 based on EU numbering system. A full chart comparing different numbering systems is provided by the International Immunogenetics Information System (“IMGT Scientific chart”). The IMGT Scientific chart refers to IgG1, the corresponding numbers in IgG2 can be readily obtained by aligning the respective sequences.

The terms “terminal β-galactose, “galactosylated glycans” or “G1, G1a, G1b, and/or G2 galactosylated glycans” refers to a glycan comprising one or two galactose molecules linked to an IgG antibody at the N-glycosylation site (Asn-297) through the N-acetylglucoseamine moieties that attach to the core mannose structure. Exemplary glycans comprising “terminal β-galactose” “galactosylated glycans” or “G1, G1a, G1b, and/or G2 galactosylated glycans” are depicted in FIGS. 1B and 1C. In some embodiments, the G1, G1a, G1b, and/or G2 galactosylated glycans may or may not contain core fucose.

The term “core fucose” or “fucosylated species” refers to a glycan comprising a fucose molecule (alpha 1-6) linked to an IgG antibody at the N-glycosylation site (Asn-297) through the N-acetylglucoseamine moieties that attach to the core mannose structure. Exemplary glycans comprising “core fucose” or “fucosylated glycans” are depicted in FIGS. 1B and 1C. In some embodiments, antibodies containing core fucose and/or a fucosylated glycans may or may not contain other glycans (including terminal β-galactose and/or high mannose glycans).

The terms “afucosylated”, “afucosylated glycans” or “afucosylation” refers to the removal or lack of a core fucose on an antibody. Exemplary afucosylated glycans are depicted in FIGS. 1B and 1C. In some embodiments, antibodies lacking core fucose may or may not contain other glycans (including terminal β-galactose and/or high mannose glycans). Afucosylated glycoforms include, but are not limited to, A1G0, A1G1a, A2G0, A2G1a, A2G1b, A2G2, and A1G1M5. See, e.g., Reusch and Tejada, Glycobiology 25(12): 1325-1334 (2015).

The term “high mannose”, “high mannose glycans” or “HM” refers to a glycan comprising more than 3 mannose molecules linked to an IgG antibody at the N-glycosylation site (Asn-297). Exemplary high mannose antibodies are depicted in FIG. 1C and Table 2, including the “Man-5 high mannose glycans” which contains two additional mannose molecules. High mannose glycans encompass glycans comprising 5, 6, 7, 8, or 9 mannose residues, abbreviated as Man 5 or M5, Man 6 or M6, Man 7 or M7, Man 8 or M8, and Man 9 or M9, respectively. Exemplary structures of Man 6, Man 7, and Man 8 are shown below.

“FcγR” or “Fc-gamma receptor” is a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

As used herein, the terms “a,” “an,” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,” and permit the presence of one or more features or components) unless otherwise noted. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used in connection with a numerical value throughout the specification and the claims denotes an interval of accuracy, familiar and acceptable to a person skilled in the art. In general, such interval of accuracy is ±10%.

### 3. Post-Translational Glycosylation and Fcγr-Mediated Cytotoxicity

3.1 Post-Translational Glycosylation

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (0 or N). For an IgG2 antibody, N-linked glycosylation occurs at Asparigine-297 (N-297) site (Eu numbering system). For panitumumab, the actual position of this Asparagine occurs at residue number 295, but in general, the N-glycosylation site is nonetheless referred to as N-297 to be consistent with the EU numbering system.

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues; (B) a complex type, which comprises more than two GlcNAc moieties and any number of other sugar types; or (C) a hybrid type, which comprises a Man residue on one side of the branch and GlcNAc at the base of a complex branch. FIG. 1A (based on Stanley et al., Chapter 8: N-Glycans, Essentials of Glycobiology, 2^{nd} ed., Cold Spring Harbor Laboratory Press; 2009) shows the three types of N-glycans.

N-linked glycans typically comprise one or more monosaccharides of galactose (Gal), N-acetylgalactosamine (GalNAc), N-acetylglucoasamine (GlcNAc), mannose (Man), N-Acetylneuraminic acid (Neu5Ac), fucose (Fuc). The commonly used symbols for such saccharides are shown in FIG. 1A.

The sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors. Controlling the glycan structure is important in recombinant production of therapeutic monoclonal antibodies, as the glycan structure attached to the Fc domain influences the interaction with the FcγRs that mediate cytotoxicity.

3.2 Glycans that Affect FcγR-Mediated Cytotoxicity

The present disclosure identifies the impact of various glycans (including, e.g., β-galactose, core-fucose, and/or high mannose) on FcγR-mediated cytotoxicity of IgG2 antibodies, such as panitumumab. Accordingly, the present disclosure provides a method of modulating Fc gamma Receptor (FcγR)-mediated cytotoxicity of an IgG2 antibody (such as panitumumab), or a composition comprising the antibody (an antibody composition). In exemplary embodiments, the method comprises modulating the amount of (a) galactosylated glycans of the antibody; (b) afucosylated glycans of the antibody; (c) high mannose glycans of the antibody; or (d) a combination thereof. Without being bound to a particular theory, it is believed that the methods disclosed herein provide means for tailor-made compositions comprising specific amounts of particular glycoforms of a given antibody, which exhibit targeted levels of FcγR-mediated cytotoxicity. Particularly relevant glycan structures are illustrated in FIG. 1C.

In exemplary aspects, the methods provided by the present disclosure relate to modulation of an IgG2 antibody (such as panitumumab), or a composition comprising the antibody (an antibody composition), wherein steps are taken to achieve a desired or predetermined level of glycoforms of the IgG2 antibody, such that the antibody or antibody composition exhibits a desired or pre-determined reference level of FcγR-mediated cytotoxicity. In exemplary embodiments, the method comprises modulating (increasing or decreasing) the amount of (a) galactosylated glycans; (b) afucosylated glycans; (c) high mannose glycans; or (d) a combination thereof of the IgG2 antibody (such as panitumumab), in order to modulate (increase or decrease) the FcγR-mediated cytotoxicity that is induced or stimulated by the antibody. In exemplary embodiments, the method comprises modulating (increasing or decreasing) the amount of glycoforms, e.g., (a) galactosylated glycoforms; (b) afucosylated glycoforms; (c) high mannose glycoforms; or (d) a combination thereof, to modulate (increase or decrease) the FcγR-mediated cytotoxicity that is induced or stimulated by the antibody.

The term “amount” when referring the amount of a particular glycan (including, e.g., (1) the amount of terminal β-galactose, (2) the amount of G1, G1a, G1b, and/or G2 galactosylated glycan, (3) the amount of core fucose, (4) the amount of fucosylated glycan, (5) the amount of afucosylated glycan, (6) the amount of high mannose glycan, and/or (7) the amount of Man-5 glycan), refers to a relative percentage of a particular glycan at the N-297 site, compared to the total amount of glycans at N-297 site. Because counting glycan species at individual molecule level is impractical/impossible, the amount of a glycan content described herein is generally calculated based on relative percentage according to commonly used analytical methods. For example, as exemplified in Example 2.2, an enzyme is used to release all N-glycans from the protein; then glycans are separated by hydrophilic interaction liquid chromatography (HILIC). HILIC results in various peaks, each peak representing a glycan species. The amount of a particular glycan is calculated as a relative percentage, based on the area of its peak, out of the total areas of all peaks. Therefore, unless otherwise specified, the amount of a glycan refers to the relative percentage of that particular glycan species, out of total N-glycans at the N-297 site, using any of the commonly used analytical method (such as HPAEC, CE-SDS, HILIC, or LC-MS).

Methods for measuring and determining the amount or relative percentage of a glycan (including, e.g., terminal β-galactose, G1, G1a, G1b, and/or G2 galactosylated glycans, core fucose, fucosylated glycans, afucosylated glycans, high mannose glycans, and/or Man-5 glycans) are well known in the art, and include, e.g., Hydrophilic Interaction Liquid Chromatography (HILIC) as described in the Examples. See also, Pace et al., Characterizing the Effect of Multiple Fc Glycan Attributes on the Effector Functions and FcγRIIIa Receptor Binding Activity of an IgG1 Antibody, Biotechnol. Prog., 2016, Vol. 32, No. 5 pages 1181-1192; and Shah, B. et al. LC-MS/MS Peptide Mapping with Automated Data Processing for Routine Profiling of N-Glycans in Immunoglobulins J. Am. Soc. Mass Spectrom. (2014) 25: 999, herein each incorporated by reference for all purposes. In some embodiments, amount can be determined or calculated as mole percent incorporation.

In some aspects, the methods disclosed herein comprise modulating the amount of terminal β-galactose, core fucose, or high mannose, or a combination thereof, attached to particular IgG2 molecules (such as panitumumab).

For example, the method may comprise increasing the amount of terminal β-galactose on an IgG2 (such as panitumumab) by, e.g., effectively changing the glycan from a G0 to a G1 or G2, or from a G1 to a G2, to increase the FcγR-mediated cytotoxicity. Alternatively, the FcγR-mediated cytotoxicity may be increased by increasing amount of antibody molecules that comprise G1, G1a, G1b, and/or G2 galactosylated glycan at the N-297 site. Also, for example, the method may comprise decreasing the amount of terminal β-galactose on an IgG2 (such as panitumumab) by, e.g., effectively changing the glycan from a G2 to a G1 or G0, or from a G1 to a G0, to decrease the FcγR-mediated cytotoxicity. Alternatively, the FcγR-mediated cytotoxicity may be decreased by decreasing amount of antibody molecules that comprise G1, G1a, G1b, and/or G2 galactosylated glycan at the N-297 site.

In other exemplary aspects, the method may comprise increasing the amount of core fucose on an IgG2 (such as panitumumab) to increase the FcγR-mediated cytotoxicity. The FcγR-mediated cytotoxicity may be increased by increasing amount of antibody molecules that comprise fucosylated glycan at the N-297 site, or by decreasing amount of antibody molecules that comprise afucosylated glycan at the N-297 site. Also, the method may comprise decreasing the amount of core fucose on an IgG2 (such as panitumumab) to decrease the FcγR-mediated cytotoxicity. The FcγR-mediated cytotoxicity may be decreased by decreasing amount of antibody molecules that comprise fucosylated glycan at the N-297 site, or by increasing amount of antibody molecules that comprise afucosylated glycan at the N-297 site.

In other exemplary aspects, the method may comprise decreasing the amount of high-mannose (e.g., Man-5) on an IgG2 (such as panitumumab) to increase the FcγR-mediated cytotoxicity. The FcγR-mediated cytotoxicity may be increased by decreasing amount of antibody molecules that comprise high-mannose glycan at the N-297 site. Also, the method may comprise increasing the amount of mannose (e.g., Man-5) on an IgG2 (such as panitumumab) to decrease the FcγR-mediated cytotoxicity. The FcγR-mediated cytotoxicity may be decreased by increasing amount of antibody molecules that comprise mannose (e.g., Man-5) at the N-297 site.

3.3 Modulating FcγR-Mediated Cytotoxicity

Fc-gamma receptors are present in two distinct classes—those that activate cells upon their crosslinking (“activation FcRs”) and those that inhibit activation upon co-engagement (“inhibitory FcRs”). In human, there are two low-affinity activation FcRs for IgG-FcγRIIa and FcγRIIIa. FcγRIIa (or FcγRIIA) is a single-chain low affinity receptor for IgG, with an ITAM sequence located in its cytoplasmic tail. It is expressed on macrophages, mast cells, monocytes, neutrophils and some B cells. It is 90% homologous in its extracellular domain to the human inhibitory FcRIIb molecule, which has an ITIM sequence in its cytoplasmic domain, expressed on B cells, macrophages, mast cells, neutrophils, monocytes but not NK cells or T cells. FcγRIIIa (or FcγRIIIA) is an oligomeric activation receptor consisting of a ligand binding a subunit and an ITAM containing gamma or zeta subunit. It is expressed on NK cells, macrophages and mast cells. It is not expressed on neutrophils, B cells or T cells. In addition, a receptor with greater than 95% sequence identity in its extracellular domain called FcRIIIb is found on human neutrophils as a GPI-anchored protein. It is capable of binding immune complexes but not activating cells in the absence of association with an ITAM containing receptor like FcRIIa. FcRII and FcRIII are about 70% identical in their ligand binding extracellular domains.

Thus, in human, IgG cytotoxic antibodies interact with four distinct low-affinity receptors—two of which are capable of activating cellular responses, FcRIIa and FcRIIIa, one of which is inhibitory, FcRIIb, and one of which will bind IgG complexes but not trigger cellular responses, FcRIIIb. Macrophages expresses FcRIIa, FcRIIb and FcRIIIa, neutrophils express FcRIIa, FcRIIb and FcRIIIb, while NK cells express only FcRIIIa. The efficacy of a therapeutic anti-tumor antibody will thus depend on the specific interactions with activation, inhibition and inert low-affinity FcRs, differentially expressed on distinct cell types.

Well-defined tumor models to study cytotoxicity of therapeutic anti-tumor antibodies are known. For example, Matui et al. described an in vitro system using A431 cells, as well as an in vivo system using A431 cell xenografts in athymic mice, to study the cytotoxicities of IgG1 and IgG2 antibodies that bind to EGFR.

In certain aspects, the FcγR-mediated cytotoxicity described herein is mediated by FcγRIIa.

In certain aspects, the FcγR-mediated cytotoxicity FcγRIIa-mediated cellular cytotoxicity.

In certain aspects, the FcγR-mediated cytotoxicity described herein is measured or determined using a FcγR reporter gene assay. In certain aspects, the reporter gene assay comprises Jurkat cells. In certain aspects, the reporter gene assay comprises a Jurkat cell expressing a FcγR receptor, a NFAT-response element, and/or a reporter gene. A reporter gene can be any gene whose expression provides a measurable signal. Exemplary reporter genes include the genes encoding green fluorescent protein (GFP), antibiotic resistance proteins (e.g., chloramphenicol transferase), toxic proteins GATA-1 DNA binding domains, colicin lysis proteins), β-galactosidase, E. coli β-galactosidase (LacZ), Halobacterium β-galactosidase, Neuropsora tyrosinase, human placental alkaline phosphatase, chloramphenicol acetyl transferase (CAT), Aequorin (jellyfish bioluminescence), Firefly luciferase (EC 1.13.12.7) form the American firefly, Photinus pyralis, Renilla luciferase (EC 1.13.12.5) from the sea pansy Renilla reniformis, and Bacterial luciferase (EC 1.14.14.3) from Photobacterium fischeri. Various other reporter genes are well known by those having ordinary skill in the art. In an exemplary embodiment, the reporter gene encodes a luciferase.

In certain aspects, the FcγR-mediated cytotoxicity described herein is measured using an ADCC assay kit. ADCC assay kits are commercially available, such as “ADCC Reporter Bioassays” by Promega (Catalog No. G7010 or G7018).

In certain aspects, the present disclosure provides a method of increasing FcγR-mediated cytotoxicity of an IgG2 antibody (such as panitumumab) or a composition comprising the antibody, as compared to a control or a reference value. In exemplary embodiments, the increase is at least or about 0.1% to about 100% increase (e.g., at least or about a 0.1% increase, at least or about a 0.2% increase, at least or about a 0.3% increase, at least or about a 0.4% increase, at least or about a 0.5% increase, at least or about a 0.55% increase, at least or about a 0.6% increase, at least or about a 0.65% increase, at least or about a 0.7% increase, at least or about a 0.75% increase, at least or about a 0.8% increase, at least or about a 0.9% increase, at least or about a 1% increase, at least or about a 1.2% increase, at least or about a 1.25% increase, at least or about a 1.3% increase, at least or about a 1.35% increase, at least or about a 1.4% increase, at least or about a 1.5% increase, at least or about a 2% increase, at least or about a 2.5% increase, at least or about a 2.7% increase, at least or about a 2.75% increase, at least or about a 2.8% increase, at least or about a 2.85% increase, at least or about a 2.9% increase, at least or about a 2.95% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, or at least or about a 100% increase), as compared to a control or a reference value. In exemplary embodiments, the increase is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900%, or at least or about 1000%, as compared to a control or a reference value. In exemplary embodiments, the FcγR-mediated cytotoxicity of the antibody, or composition comprising the antibody, increases by at least about 1.1-fold, at least about 1.2-fold, at least about 1.3-fold, at least about 1.4-fold, at least about 1.5-fold, at least about 1.6-fold, at least about 1.7-fold, at least about 1.8-fold, or at least about 1.9-fold, as compared to a control or a reference value. In exemplary embodiments, the FcγR-mediated cytotoxicity of the antibody, or composition comprising the antibody, increases by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, relative to a control or a reference value. In exemplary embodiments, the FcγR-mediated cytotoxicity of the antibody, or composition comprising the antibody, increases by from about 1.1-fold to about 10-fold, from about 1.2-fold to about 10-fold, from about 1.3-fold to about 10-fold, from about 1.4-fold to about 10-fold, from about 1.5-fold to about 10-fold, from about 1.1-fold to about 5-fold, from about 1.2-fold to about 5-fold, from about 1.3-fold to about 5-fold, from about 1.4-fold to about 5-fold, or from about 1.5-fold to about 5-fold, as compared to a control or a reference value.

In certain aspects, the present disclosure provides a method of decreasing FcγR-mediated cytotoxicity of an IgG2 antibody (such as panitumumab) or a composition comprising the antibody, as compared to a control or a reference value. In exemplary embodiments, the decrease is at least or about 0.1% to about 100% decrease (e.g., at least or about a 0.1% decrease, at least or about a 0.2% decrease, at least or about a 0.3% decrease, at least or about a 0.4% decrease, at least or about a 0.5% decrease, at least or about a 0.55% decrease, at least or about a 0.6% decrease, at least or about a 0.65% decrease, at least or about a 0.7% decrease, at least or about a 0.75% decrease, at least or about a 0.8% decrease, at least or about a 0.9% decrease, at least or about a 1% decrease, at least or about a 1.2% decrease, at least or about a 1.25% decrease, at least or about a 1.3% decrease, at least or about a 1.35% decrease, at least or about a 1.4% decrease, at least or about a 1.5% decrease, at least or about a 2% decrease, at least or about a 2.5% decrease, at least or about a 2.7% decrease, at least or about a 2.75% decrease, at least or about a 2.8% decrease, at least or about a 2.85% decrease, at least or about a 2.9% decrease, at least or about a 2.95% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, or at least or about a 100% decrease), as compared to a control or a reference value. In exemplary embodiments, the decrease is over 100%, e.g., at least or about 125%, at least or about 150%, at least or about 175%, at least or about 200%, at least or about 300%, at least or about 400%, at least or about 500%, at least or about 600%, at least or about 700%, at least or about 800%, at least or about 900%, or at least or about 1000%, as compared to a control or a reference value. In exemplary embodiments, the FcγR-mediated cytotoxicity of the antibody, or composition comprising the antibody, decreases by at least about 1.1-fold, at least about 1.2-fold, at least about 1.3-fold, at least about 1.4-fold, at least about 1.5-fold, at least about 1.6-fold, at least about 1.7-fold, at least about 1.8-fold, or at least about 1.9-fold, as compared to a control or a reference value. In exemplary embodiments, the FcγR-mediated cytotoxicity of the antibody, or composition comprising the antibody, decreases by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, as compared to a control or a reference value. In exemplary embodiments, the FcγR-mediated cytotoxicity of the antibody, or composition comprising the antibody, decreases by from about 1.1-fold to about 10-fold, from about 1.2-fold to about 10-fold, from about 1.3-fold to about 10-fold, from about 1.4-fold to about 10-fold, from about 1.5-fold to about 10-fold, from about 1.1-fold to about 5-fold, from about 1.2-fold to about 5-fold, from about 1.3-fold to about 5-fold, from about 1.4-fold to about 5-fold, or from about 1.5-fold to about 5-fold, as compared to a control or a reference value.

As used herein, the “control” or “reference value” here is the level of FcγR-mediated cytotoxicity of the antibody, or a composition comprising the antibody, prior to an experimental intervention directed at modulating the glycan profile (such as the level of cytotoxicity when first measured). If an antibody, or a composition comprising the antibody, has undergone experimental intervention directed at modulating the glycan profile, but additional modulation is desired, then the “control” or “reference value” can be the level of FcγR-mediated cytotoxicity prior to any additional experimental intervention directed at further modulating the glycan profile.

In certain aspects, the reference value is the level of the FcγR-mediated cytotoxicity exhibited by commercially available panitumumab samples at the same dose (e.g., same amount of antibody molecules). In certain aspects, the reference value is a pre-determined level that provides therapeutic benefit.

In certain aspects, the present disclosure provides a method comprising modulating (i.e. increasing or decreasing) the amount of a specific glycan species (e.g., galactosylated glycans, G1, G1a, G1b, and/or G2 galactosylated glycans, fucosylated glycans, afucosylated glycans, core fucose, high mannose glycans, Man-5 glycans, or a combination thereof) of the antibody to a total amount of at least or about 0.5%, at least or about 1%, at least or about 2%, at least or about 3%, at least or about 5%, at least or about 7%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, from about 0.5% to about 98%, from about 0.5% to about 98%, from about 0.5% to about 98%, from about 0.1% to about 99%, from about 0.5% to about 98%, from about 0.5% to about 95%, from about 1% to about 90%, from about 1% to about 85%, from about 5% to about 85%, from about 10% to about 85%, or from about 10% to about 80%. As described above, the percentage, when describing specific glycan species, generally refers to the relative percentage of a particular glycan species, out of total glycan content at the N-297 site, calculated according to any of the art-recognized analytical methods (such as HILIC, LC-MS). In one exemplary embodiment, the relative percentage is calculated according to the areas of chromatographic peaks.

In certain aspect, the disclosure provides a method of modulating Fc gamma Receptor (FcγR)-mediated cytotoxicity of panitumumab, comprising increasing or decreasing the amount of terminal β-galactose at the N-297 glycosylation site of panitumumab, or increasing or decreasing the amount of panitumumab molecules that comprise G1, G1a, G1b, and/or G2 galactosylated glycan at the N-297 site.

In certain embodiments, the disclosure provides a method of increasing Fc gamma Receptor (FcγR)-mediated cytotoxicity of panitumumab, comprising increasing the amount of terminal β-galactose at the N-297 glycosylation site of panitumumab, or increasing the amount of panitumumab molecules that comprise G1, G1a, G1b, and/or G2 galactosylated glycan at the N-297 site. In certain embodiments, an increase of about 1 percent of β-galactose increases FcγR-mediated cytotoxicity by from about 0.55 percent to about 0.75 percent, such as about 0.55 percent, about 0.6 percent, about 0.65 percent, about 0.7 percent, or about 0.75 percent. As described above, the percentages of β-galactose, or G1, G1a, G1b, G2 galactosylated glycans, refer to relative percentages of the respective glycan species, out of the total glycan content at the N-297 site.

When quantifying the relationship between FcγR-mediated cytotoxicity and various glycans (e.g., change of the percentage level of a particular glycan, and the corresponding changes in cytotoxicity level), the FcγR-mediated cytotoxicity is often expressed as a relative value, quantified against a standard. For example, “percent relative activity” (against a standard) can be used to express FcγR-mediated cytotoxicity level. “Percent relative activity” can be calculated as: (i) cytotoxic activity of the sample/cytotoxic activity of the standard (“/” means divide); or (ii) cytotoxic activity of the standard/cytotoxic the activity of sample (“1” means divide). For example, if sample A exhibits 50% cytotoxicity level, as compared to a standard, and sample B exhibits 51% cytotoxicity level, as compared to the same standard, then it can be said that the FcγR-mediated cytotoxicity is increased by 1% from sample A to sample B.

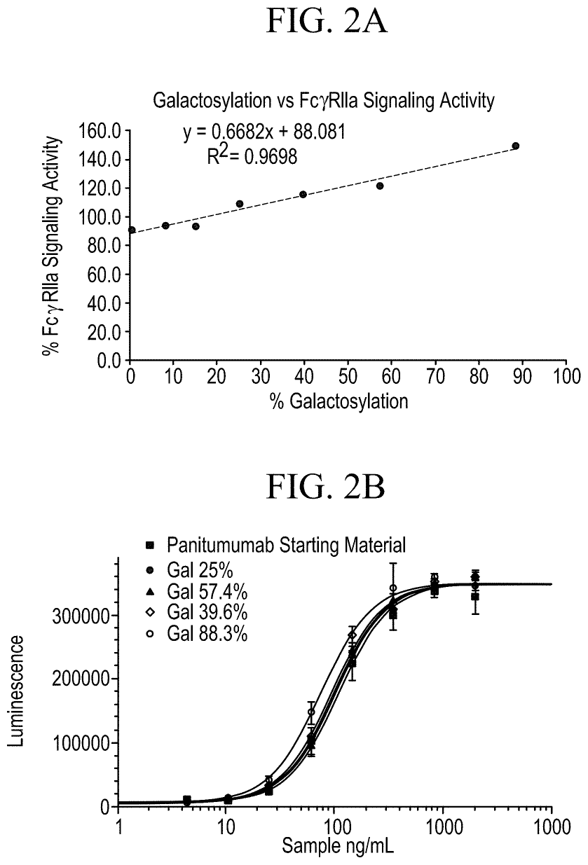

In certain embodiments, the standard is the level of the FcγR-mediated cytotoxicity exhibited by commercially available panitumumab samples at the same dose (e.g., same amount of antibody molecules). Therefore, in certain embodiments, a quantitative relationship is established using relative cytotoxicity level. For example, referring to FIG. 2A, when terminal β-galactose is about 0%, the relative cytotoxicity level (calculated against commercially available panitumumab samples at the same dose) is about 88%. When terminal β-galactose is increased to about 10%, the relative cytotoxicity level (calculated against commercially available panitumumab samples at the same dose) is about 95%. Thus, an increase of about 1 percent of terminal β-galactose correlates to an increase of FcγR-mediated cytotoxicity by about 0.67 percent. This means that for every 1 percent increase of terminal β-galactose, the relative cytotoxicity level of the panitumumab sample increases by 0.67 percent.

In certain embodiments, the relative cytotoxicity level can be calculated based on EC50 values measured in a bioassay. For example, if a reporter gene is used to determine the EC50 of the cytotoxicity exhibited by a sample antibody, then the relative cytotoxicity level can be calculated as EC50 sample/EC50 standard, or EC50 standard/EC50 sample (“/” means divide).

If preferred, the relative cytotoxicity value of a sample can be measured multiple times (e.g., twice, three times, four times), and the result can be reported as the mean of these multiple values.

In certain embodiments, the disclosure provides a method of decreasing Fc gamma Receptor (FcγR)-mediated cytotoxicity of panitumumab, comprising decreasing the amount of terminal β-galactose at the N-297 glycosylation site of panitumumab, or decreasing the amount of panitumumab molecules that comprise G1, G1a, G1b, and/or G2 galactosylated glycan at the N-297 site. In certain embodiments, a decrease of about 1 percent of β-galactose decreases FcγR-mediated cytotoxicity by from about 0.55 percent to about 0.75 percent, such as about 0.55 percent, about 0.6 percent, about 0.65 percent, about 0.7 percent, or about 0.75 percent. Again, changes in cytotoxicity level is generally calculated based on relative cytotoxicity value as described above.

In certain aspects, the disclosure provides a method of modulating Fc gamma Receptor (FcγR)-mediated cytotoxicity of panitumumab, comprising increasing or decreasing the amount of panitumumab molecules that comprise fucosylated glycan at the N-297 site, or increasing or decreasing the amount of panitumumab molecules that comprise afucosylated glycan at the N-297 site

In certain embodiments, the disclosure provides a method of increasing Fc gamma Receptor (FcγR)-mediated cytotoxicity of panitumumab, comprising increasing the amount of panitumumab molecules that comprise fucosylated glycan at the N-297 site. In certain embodiments, an increase of about 1 percent of fucosylated panitumumab molecules increases FcγR mediated cytotoxicity by from about 2.70 percent to about 3.0 percent, such as about 3.0 percent, about 2.95 percent, about 2.90 percent, about 2.85 percent, or about 2.70 percent. In certain embodiments, the disclosure provides a method of increasing Fc gamma Receptor (FcγR)-mediated cytotoxicity of panitumumab, comprising decreasing the amount of panitumumab molecules that comprise afucosylated glycan at the N-297 site. In certain embodiments, a decrease of about 1 percent of afucosylated panitumumab molecules increases FcγR mediated cytotoxicity by from about 2.70 percent to about 3.0 percent, such as about 3.0 percent, about 2.95 percent, about 2.90 percent, about 2.85 percent, or about 2.70 percent. Again, changes in cytotoxicity level is generally calculated based on relative cytotoxicity value as described above.

In certain embodiments, the disclosure provides a method of decreasing Fc gamma Receptor (FcγR)-mediated cytotoxicity of panitumumab, comprising decreasing the amount of panitumumab molecules that comprise fucosylated glycan at the N-297 site. In certain embodiments, a decrease of about 1 percent of fucosylated panitumumab molecules increases FcγR mediated cytotoxicity by from about 2.70 percent to about 3.0 percent, such as about 3.0 percent, about 2.95 percent, about 2.90 percent, about 2.85 percent, or about 2.70 percent. In certain embodiments, the disclosure provides a method of decreasing Fc gamma Receptor (FcγR)-mediated cytotoxicity of panitumumab, comprising increasing the amount of panitumumab molecules that comprise afucosylated glycan at the N-297 site. In certain embodiments, an increase of about 1 percent of afucosylated panitumumab molecules decreases FcγR mediated cytotoxicity by from about 2.70 percent to about 3.0 percent, such as about 3.0 percent, about 2.95 percent, about 2.90 percent, about 2.85 percent, or about 2.70 percent. Again, changes in cytotoxicity level is generally calculated based on relative cytotoxicity value as described above.

In exemplary embodiments, the fucosylated glycans modulated (increased or decreased) on the antibody include one or more of the fucosylated glycans selected from the group consisting of: A1G0, A1G1, A2G0, A2G1a, A2G1b, A2G2, and A1G1M5. In exemplary embodiments, the afucosylated glycans modulated (increased or decreased) on the antibody include one or more of the afucosylated glycans selected from the group consisting of: A1G0, A1G1, A2G0, A2G1a, A2G1b, A2G2, and A1G1M5.

In certain aspects, the disclosure provides a method of modulating Fc gamma Receptor (FcγR)-mediated cytotoxicity of panitumumab, comprising increasing or decreasing the amount of panitumumab molecules that comprise a high-mannose glycan at the N-297 site. In exemplary embodiments, the high mannose glycan can be Man-5, Man-6, Man-7, Man-8, or Man-9. In exemplary embodiments, the high mannose glycan is Man-5.

In certain aspects, the disclosure provides a method of increasing Fc gamma Receptor (FcγR)-mediated cytotoxicity of panitumumab, comprising decreasing the amount of panitumumab molecules that comprise a high-mannose glycan at the N-297 site. In certain embodiments, a decrease of about 1 percent of high-mannose glycan increases FcγR-mediated cytotoxicity by from about 1.2 percent to about 1.4 percent, such as about 1.2 percent, about 1.25 percent, about 1.3 percent, about 1.35 percent, or about 1.40 percent. Again, changes in cytotoxicity level is generally calculated based on relative cytotoxicity value as described above. In certain embodiments, the high-mannose is Mannose-5 (Man-5).

In certain aspects, the disclosure provides a method of decreasing Fc gamma Receptor (FcγR)-mediated cytotoxicity of panitumumab, comprising increasing the amount of panitumumab molecules that comprise a high-mannose glycan at the N-297 site. In certain embodiments, an increase of about 1 percent of high-mannose glycan decreases FcγR-mediated cytotoxicity by from about 1.2 percent to about 1.4 percent, such as about 1.2 percent, about 1.25 percent, about 1.3 percent, about 1.35 percent, or about 1.40 percent. Again, changes in cytotoxicity level is generally calculated based on relative cytotoxicity value as described above. In certain embodiments, the high-mannose is Mannose-5 (Man-5).

The methods provided herein also include methods of matching the FcγR-mediated cytotoxicity of an IgG2 antibody sample (such as a panitumumab sample) to a reference value, by modulating the amount of glycans (e.g., galactosylated glycans, terminal β-galactose, G1, G1a, G1b, and/or G2 galactosylated glycans, fucosylated glycans, afucosylated glycans, core fucose, high mannose glycans, Man-5 glycans, or a combination thereof) in the sample antibody to match the reference value. In certain aspects, the reference value is the level of the FcγR-mediated cytotoxicity exhibited by commercially available panitumumab samples at the same dose (e.g., same amount of antibody molecules). In certain aspects, the reference value is a pre-determined level that provides therapeutic benefit. In exemplary embodiments, the method comprises measuring the cytotoxic activity of the sample antibody and/or a reference sample using the methods described herein. In exemplary aspects, determining or measuring the cytotoxic activity of the antibody sample and/or reference sample occurs: (i) before modulating the amount of glycans in the antibody, (ii) after modulating the amount of glycans in the antibody; or (iii) before and after modulating the amount of glycans in the antibody.

In certain aspects, the disclosure provides a method of matching the Fc gamma Receptor (FcγR)-mediated cytotoxicity of an IgG2 antibody sample (such as a panitumumab sample) to a reference value, comprising: (1) obtaining a reference value of FcγR-mediated cytotoxicity; (2) determining the FcγR-mediated cytotoxicity of said IgG2 antibody sample (such as panitumumab sample); and (3) changing the FcγR-mediated cytotoxicity of said IgG2 antibody sample (such as panitumumab sample) by increasing or decreasing the amount of terminal β-galactose at the N-297 glycosylation site of the antibody, or increasing or decreasing the amount of IgG2 molecules that comprise G1, G1a, G1b, and/or G2 galactosylated glycan at the N-297 site; such that the difference in FcγR-mediated cytotoxicity between the antibody sample and the reference value is about 35% or less. In certain embodiments, the difference in FcγR-mediated cytotoxicity between the IgG2 antibody sample (such as a panitumumab sample) and the reference value is about 30% or less, about 25% or less, about 20% or less, about 15% or less, about 10% or less, or about 5% or less. In some instances, step (1) (“obtaining a reference value of FcγR-mediated cytotoxicity”) occurs before, after or at the same time as step (2) (“determining the FcγR-mediated cytotoxicity of said IgG2 sample or panitumumab sample”) and/or step (3) (“changing the FcγR-mediated cytotoxicity of said IgG2 sample or panitumumab sample”); while in other instances, step (2) occurs before, after or at the same time as step (1) and/or step (3).

In certain aspects, the FcγR-mediated cytotoxicity of the IgG2 sample or panitumumab sample is increased by increasing the amount of terminal β-galactose at the N-297 glycosylation site of the antibody, or increasing the amount of panitumumab molecules that comprise G1, G1a, G1b, and/or G2 galactosylated glycan at the N-297 site. In certain embodiments, an increase of about 1 percent of β-galactose increases FcγR-mediated cytotoxicity by from about 0.55 percent to about 0.75 percent, such as about 0.55 percent, about 0.6 percent, about 0.65 percent, about 0.7 percent, or about 0.75 percent. The calculations of glycan level and cytotoxicity level are described above, and in general changes in cytotoxicity level is generally calculated based on relative cytotoxicity value as described above.

In certain aspects, the FcγR-mediated cytotoxicity of the IgG2 sample or panitumumab sample is decreased by decreasing the amount of terminal β-galactose at the N-297 glycosylation site of the antibody, or decreasing the amount of antibody molecules that comprise G1, G1a, G1b, and/or G2 galactosylated glycan at the N-297 site. In certain embodiments, a decrease of about 1 percent of β-galactose increases FcγR-mediated cytotoxicity by from about 0.55 percent to about 0.75 percent, such as about 0.55 percent, about 0.6 percent, about 0.65 percent, about 0.7 percent, or about 0.75 percent. Again, changes in cytotoxicity level is generally calculated based on relative cytotoxicity value as described above.

In certain aspect, the disclosure provides a method of matching the Fc gamma Receptor (FcγR)-mediated cytotoxicity of an IgG2 antibody sample (such as a panitumumab sample) to a reference value, comprising: (1) obtaining a reference value of FcγR-mediated cytotoxicity; (2) determining the FcγR-mediated cytotoxicity of said IgG2 antibody sample (such as panitumumab sample); and (3) changing the FcγR-mediated cytotoxicity of said IgG2 antibody sample (such as panitumumab sample) by increasing or decreasing the amount of IgG2 molecules that comprise fucosylated glycan at the N-297 site, or by increasing or decreasing the amount of IgG2 molecules that comprise afucosylated glycan at the N-297 site; such that the difference in FcγR-mediated cytotoxicity between the antibody sample and the reference value is about 35% or less. In certain embodiments, the difference in FcγR-mediated cytotoxicity between the IgG2 antibody sample (such as a panitumumab sample) and the reference value is about 30% or less, about 25% or less, about 20% or less, about 15% or less, about 10% or less, or about 5% or less. In some instances, step (1) (“obtaining a reference value of FcγR-mediated cytotoxicity”) occurs before, after or at the same time as step (2) (“determining the FcγR-mediated cytotoxicity of said IgG2 sample or panitumumab sample”) and/or step (3) (“changing the FcγR-mediated cytotoxicity of said IgG2 sample or panitumumab sample”); while in other instances, step (2) occurs before, after or at the same time as step (1) and/or step (3).

In certain aspects, the FcγR-mediated cytotoxicity of the IgG2 sample or panitumumab sample is increased by increasing the amount of panitumumab molecules that comprise fucosylated glycan at the N-297 site, or by decreasing the amount of panitumumab molecules that comprise afucosylated glycan at the N-297 site. In certain embodiments, an increase of about 1 percent of fucosylated panitumumab molecules increases FcγR mediated cytotoxicity by from about 2.7 percent to about 3.0 percent, such as about 3.0 percent, about 2.95 percent, about 2.90 percent, about 2.85 percent, or about 2.70 percent. In certain embodiments, a decrease of about 1 percent of afucosylated panitumumab molecules increases FcγR mediated cytotoxicity by from about 2.7 percent to about 3.0 percent, such as about 3.0 percent, about 2.95 percent, about 2.90 percent, about 2.85 percent, or about 2.70 percent. Again, changes in cytotoxicity level is generally calculated based on relative cytotoxicity value as described above.

In certain aspects, the FcγR-mediated cytotoxicity of the IgG2 sample or panitumumab sample is decreased by decreasing the amount of panitumumab molecules that comprise fucosylated glycan at the N-297 site, or by increasing the amount of panitumumab molecules that comprise afucosylated glycan at the N-297 site. In certain embodiments, a decrease of about 1 percent of fucosylated panitumumab molecules decreases FcγR mediated cytotoxicity by from about 2.7 percent to about 3.0 percent, such as about 3.0 percent, about 2.95 percent, about 2.90 percent, about 2.85 percent, or about 2.70 percent. In certain embodiments, an increase of about 1 percent of afucosylated panitumumab molecules decreases FcγR mediated cytotoxicity by from about 2.7 percent to about 3.0 percent, such as about 3.0 percent, about 2.95 percent, about 2.90 percent, about 2.85 percent, or about 2.70 percent. Again, changes in cytotoxicity level is generally calculated based on relative cytotoxicity value as described above.

In certain aspect, the disclosure provides a method of matching the Fc gamma Receptor (FcγR)-mediated cytotoxicity of an IgG2 antibody sample (such as a panitumumab sample) to a reference value, comprising: (1) obtaining a reference value of FcγR-mediated cytotoxicity; (2) determining the FcγR-mediated cytotoxicity of said IgG2 antibody sample (such as panitumumab sample); and (3) changing the FcγR-mediated cytotoxicity of said IgG2 antibody sample (such as panitumumab sample) by increasing or decreasing the amount of IgG2 molecules that comprise high-mannose glycan at the N-297 site; such that the difference in FcγR-mediated cytotoxicity between the antibody sample and the reference value is about 35% or less. In certain embodiments, the difference in FcγR-mediated cytotoxicity between the IgG2 antibody sample (such as a panitumumab sample) and the reference value is about 30% or less, about 25% or less, about 20% or less, about 15% or less, about 10% or less, or about 5% or less. In some instances, step (1) (“obtaining a reference value of FcγR-mediated cytotoxicity”) occurs before, after or at the same time as step (2) (“determining the FcγR-mediated cytotoxicity of said IgG2 sample or panitumumab sample”) and/or step (3) (“changing the FcγR-mediated cytotoxicity of said IgG2 sample or panitumumab sample”); while in other instances, step (2) occurs before, after or at the same time as step (1) and/or step (3).

In certain aspects, the FcγR-mediated cytotoxicity of the IgG2 sample or panitumumab sample is increased by decreasing the amount of panitumumab molecules that comprise high-mannose glycan at the N-297 site. In certain embodiments, a decrease of about 1 percent of high-mannose glycan increases FcγR-mediated cytotoxicity by from about 1.2 percent to about 2.4 percent, such as about 1.2 percent, about 1.25 percent, about 1.3 percent, about 1.35 percent, or about 1.40 percent. Again, changes in cytotoxicity level is generally calculated based on relative cytotoxicity value as described above.

In certain aspects, the FcγR-mediated cytotoxicity of the IgG2 sample or panitumumab sample is decreased by increasing the amount of panitumumab molecules that comprise high-mannose glycan at the N-297 site. In certain embodiments, an increase of about 1 percent of high-mannose glycan decreases FcγR-mediated cytotoxicity by from about 1.2 percent to about 2.4 percent, such as about 1.2 percent, about 1.25 percent, about 1.3 percent, about 1.35 percent, or about 1.40 percent. Again, changes in cytotoxicity level is generally calculated based on relative cytotoxicity value as described above.

3.4 Methods of Modulating Glycans

Suitable methods of modulating the amount of glycans, such as galactosylated glycans (including, e.g., terminal β-galactose, G1, G1a, G1b, and/or G2 galactosylated glycans), afucosylated glycans, fucosylated or glycans containing core fucose, and/or high mannose glycans (including, e.g., Man-5 glycan), on glycoproteins, including antibodies, are known in the art. See, e.g., Zhang et al., Drug Discovery Today 21(5): 2016). Thus, in some aspects, glycosylation-competent cells—which can be used to recombinantly produce a glycoprotein, including antibodies—are cultured under particular conditions to achieve the desired level of glycans.

For example, International Patent Publication Nos. WO 2013/114164; WO 2013/114245; WO 2013/114167; WO 2015128793; and WO 2016/089919 each teach recombinant cell culturing techniques useful to modulate glycans, such as galactosylated glycans (including, e.g., terminal β-galactose or G1, G1a, G1b, and/or G2 galactosylated glycans), afucosylated glycans, fucosylated glycans or glycans containing core fucose, and/or high mannose glycans (including, e.g., Man-5 glycans), including: methods of obtaining glycoproteins having increased percentage of total afucosylated glycans (WO 2013/114164); methods of obtaining glycoproteins having increased percentage of Man5 glycans and/or afucosylated glycans (WO 2013/114245); methods of obtaining glycoproteins having specific amounts of high mannose glycans, afucosylated glycans and G0F glycans (WO 2013/114167); methods of obtaining glycoproteins having high-mannose glycan and reduced galactosylation and/or high galactosylated glycans (WO 2015128793); and methods of manipulating the fucosylated glycan content on a recombinant protein (WO 2016/089919). The cell culture methods described by WO 2013/114164; WO 2013/114245; WO 2013/114167; WO 2015128793; and WO 2016/089919 include modifying one or more cell culture parameters such as temperature, pH, culturing cells with manganese ion or salts thereof (e.g., 0.35 μM to about 20 μM Manganese) and/or culturing cells with copper (e.g., 10 to 100) and manganese (e.g., 50 to 1000 nM) to modulate specific glycans.

Additionally, International Patent Publication No. WO 2015/140700 describes culturing cells in the presence of betaine to increase afucosylated glycans, or culturing cells with manganese, galactose and betaine to obtain target values of mannosylated, galactosylated and afucosylated glycans. U.S. Patent Application Publication No. 2014/0356910 teaches methods of increasing high mannose glycoforms by manipulating the mannose to total hexose ratio in the cell culture media formulation. Pacis et al., Biotechnology and Bioengineering 108(10): 2348-2358 (2011) teaches obtaining high levels of Man5 glycans by increasing cell culture medium osmolality levels and extending culture duration. Similarly, Konno et al., Cytotechnology 64: 249-3+6 (2012) describes methods of controlling antibody fucose content through culture medium osmolality. Wong et al., Biotechnology and Bioengineering 89(2): 164-177 (2004) teaches methods of decreasing recombinant protein sialylation and increasing high mannose glycans by using low glutamine fed-batch cultures. International Patent Publication No. WO 2017/079165 describes methods of increasing or decreasing afucosylated or fucosylated forms of recombinant proteins by using host cells genetically modified to have no GMD or FX and culturing the host cell with fucose. International Patent Publication No. WO 2017/134667 describes culturing cells with nicotinamide and fucose to produce antibodies having decreased levels of afucosylation. Sha et al., TIBs 34(10): 835-846 (2016) also reviews several methods of modulating glycans, including, for example, culturing with uridine, manganese, and galactose to increase galactosylation levels on antibodies, and using mannose as a carbon source to increase high mannose glycoforms.

Accordingly, the methods of the present disclosure, in exemplary aspects, comprises adopting one or more of the practices, cell culture media and/or cell culture conditions taught in any one or more of the above references or other reference described herein, in order to modulate the amounts of the galactosylated glycans (including, e.g., terminal β-galactose or G1, G1a, G1b and/or G2 galactosylated glycans), afucosylated glycans, fucosylated glycans or glycans containing core fucose, and/or high mannose glycans (including, e.g., Man-5 glycans). In exemplary aspects, the method comprises culturing glycosylation-competent cells expressing the antibody in a cell culture medium under conditions which modulate the level(s) of the galactosylated glycans (including, e.g., terminal β-galactose or G1, G1a, G1b and/or G2 galactosylated glycans), afucosylated glycans, fucosylated glycans or glycans containing core fucose, and/or high mannose glycans (including, e.g., M5 high mannose species). For example, the method, in some aspects, comprises culturing glycosylation-competent cells expressing the antibody in a cell culture medium under conditions which modulate the level(s) of the glycan(s), wherein the cell culture medium comprises fucose or fucose and glucose.

In the methods comprising maintaining or culturing cells in cell culture, the cell culture may be maintained according to any set of conditions suitable for a recombinant glycosylated protein or antibody production. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the method comprises culturing glycosylation-competent cells expressing the antibody in a cell culture medium under conditions which modulate the level(s) of the glycan(s), wherein the osmolality of the cell culture medium is increased to decrease the level of afucosylated glycans of the antibody, e.g., as taught by Konno et al., supra. In exemplary aspects, the method comprises culturing glycosylation-competent cells expressing the antibody in a cell culture medium under conditions which modulate the level(s) of the glycan(s), wherein the pH and the temperature of the cell culture are adjusted, e.g., as taught by WO 2013/114164, WO 2013/114245, WO 2013/114167, or WO 2015/128793, each herein incorporated by reference.

In exemplary aspects, the methods of the disclosure comprise maintaining the glycosylation-competent cells in a cell culture medium at a pH, temperature, osmolality, and dissolved oxygen level suitable for recombinant glycosylated protein or antibody production, as well-known in the art. In exemplary aspects, the cell culture is maintained in a medium suitable for cell growth and/or is provided with one or more feeding media according to any suitable feeding schedule as well-known in the art.

In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

In additional or alternative aspects, the glycosylation-competent cells which recombinantly produce the antibody are genetically modified in a way to modulate the glycans (such as the galactosylated glycans (including, e.g., terminal β-galactose or G1, G1a, G1b and/or G2 galactosylated species), afucosylated glycans or glycans containing core fucose, and/or high mannose glycans (including, e.g., M5 high mannose species) of the antibody. In exemplary aspects, the glycosylation-competent cells are genetically modified to alter activity of an enzyme of the de novo pathway or the salvage pathway. Optionally, the glycosylation-competent cells are genetically modified to knock-out a gene encoding GDP-keto-6-deoxymannonse-3,5-epimerase, 4-reductase. In exemplary embodiments, the glycosylation-competent cells are genetically modified to alter the activity of an enzyme of the de novo pathway or the salvage pathway. These two pathways of fucose metabolism are well-known in the art and shown in FIG. 1E. In exemplary embodiments, the glycosylation-competent cells are genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are genetically modified to knock-out a gene encoding FX. In exemplary embodiments, the glycosylation-competent cells are genetically modified to alter the activity β(1,4)-N-acetylglucosaminyltransferase III (GNTIII) and/or GDP-6-deoxy-D-lyxo-4-hexulose reductase (RMD). In exemplary aspects, the glycosylation-competent cells are genetically modified to overexpress GNTIII and/or RMD. In exemplary embodiments, the glycosylation-competent cells are genetically modified to have altered beta-galactosyltransferase activity. In some embodiments, the glycosylation-competent cells are genetically modified to modulate the expression level of the gene encoding GDP-keto-6-deoxymannonse-3,5-epimerase, 4-reductase, β1-4 galactosyltransferase, and/or β1-4 N-acetylgalactosaminyltransferase.

Several ways are known in the art for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing β-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli.

In exemplary aspects, targeted glycan amounts are achieved through post-production chemical or enzyme treatment of the antibody. In exemplary aspects, the method of the present disclosure comprises treating the antibody with a chemical or enzyme after the antibody is recombinantly produced. In exemplary aspects, the chemical or enzyme is selected from the group consisting of EndoS; Endo-S2; Endo-D; Endo-M; endoLL; α-fucosidase; β-(1-4)-Galactosidase; Endo-H; Endo F1; Endo F2; Endo F3; β-1,4-galactosyltransferase; kifunensine, and PNGase F. In exemplary aspects, the chemical or enzyme is incubated with the antibody at various times to generate antibodies having different amounts of glycans. In some aspects, the antibody is incubated with β-1,4-galactosyltransferase as described in the Examples. In some additional aspects, antibodies having different levels of galactose can be generated by incubating the antibody with β-1,4-galactosyltransferase fora set period of time, including, but not limited to, about 10 minutes, about 20 minutes, about 30 minutes, about 1 hour, about 2 hours, about 4 hours, about 9 hours or for a period of time falling in the range between about 10 minutes and about 9 hours.

3.5 Methods of Measuring Glycans

Various methods are known in the art for assessing glycoforms present in a glycoprotein-containing composition, including antibodies, or for determining, detecting or measuring a glycoform profile of a particular sample comprising glycoproteins. Suitable methods include, but are not limited to, Hydrophilic Interaction Liquid Chromatography (HILIC), Liquid chromatography-tandem mass spectrometry (LC-MS), positive ion MALDI-TOF analysis, negative ion MALDI-TOF analysis, HPLC, weak anion exchange (WAX) chromatography, normal phase chromatography (NP-HPLC), exoglycosidase digestion, Bio-Gel P-4 chromatography, anion-exchange chromatography and one-dimensional NMR spectroscopy, and combinations thereof. See, e.g., Pace et al., Biotechnol. Prog., 2016, Vol. 32, No. 5 pages 1181-1192; Shah, B. et al. J. Am. Soc. Mass Spectrom. (2014) 25: 999; Mattu et al., JBC 273: 2260-2272 (1998); Field et al., Biochem J 299 (Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481; Kurogochi et al., PLOS One 10(7): e0132848; doi:10.1371/journal.pone.0132848; Thomann et al., PLOS One 10(8): e0134949. Doi:10.1371/journal.pone.0134949; Pace et al., Biotechnol. Prog. 32(5): 1181-1192 (2016); and Geoffrey, R. G. et. al. Analytical Biochemistry 1996, Vol. 240, pages 210-226. Also, the examples set forth herein describe a suitable method for assessing glycoforms present in a glycoprotein containing composition such as an antibody.

For example, glycan content can be measured by high pH anion exchange chromatography (HPAEC), as described in Wuhrer et al. (Journal of Chromatography B Vol. 825:124-133, 2005) and Dell et al. (Science Vol. 291:2351-2356). Briefly, N-glycans are removed enzymatically from the recombinant glycoproteins, such as a recombinant monoclonal antibody, and labeled with a fluorescent tag (e.g., 2-Aminobenzamide or 2-aminobenzoic acid) at the reducing terminus. The fluorescent N-glycans are separated by HPAEC, and detected using fluorescence detection. Separation of the neutral N-glycans is generally based on the increasing complexity in the N-glycan structures. Separation of the charged N-glycans is based on the number and type of sialic acid, sulfate, or other modifications present from which a charge number can be derived. These glycan profiles of test samples are compared visually to an appropriate standard.

Example 2.2 uses Hydrophilic Interaction Liquid Chromatography (HILIC). Briefly, the glycan species can be analyzed based on the following steps: (i) release of the N-glycans (e.g., by an enzyme such as PNGase F), (ii) labeling (e.g., with 2-aminobenzoic acid or 2-aminobenzamide), (iii) removal of the free label (e.g., by gel filtration or solid-phase extraction); (iv) separation of glycan species by HILIC; and (v) detection (e.g., by fluorescence spectrometry). Additional details of HILIC is provided by Melmer et. al., Analytical and Bioanalytical Chemistry, September 2010, Volume 398, Issue 2, pp 905-914.

Another commonly used method is liquid chromatography-tandem mass spectrometry (LC-MS). After the release of the N-glycans, labeling, and removal of free label, the samples can be analyzed by techniques that combine the physical separation capabilities of liquid chromatography (or HPLC) with the mass analysis capabilities of mass spectrometry (MS). See, e.g., Wang et. al., Biotech Method, 17 Jan. 2018, doi.org/10.1002/biot.201700185.

3.6 Antibody Compositions

Provided herein are also compositions comprising recombinant glycosylated proteins and antibodies produced by the methods described herein. In exemplary embodiments, the compositions are prepared by methods which modulate the amount of glycans (e.g., galactosylated glycans, terminal β-galactose, G1, G1a, G1b and/or G2 galactosylated glycans, afucosylated glycans, fucosylated glycans, core fucose, high mannose glycans, Man-5 glycans, or a combination thereof) in an antibody. In exemplary aspects, the recombinant glycosylated protein is an IgG2 antibody, such as panitumumab. Accordingly, antibody compositions are provided herein, including IgG2 antibodies (e.g., panitumumab) having increased or decreased FcγR-mediated cytotoxicity, wherein the IgG2 antibody (e.g., panitumumab) have been engineered to have increased or decreased FcγR-mediated cytotoxicity, as compared to a control or reference value, by modulating glycan profiles as described above.

In exemplary embodiments, the antibody compositions provided herein are combined with a pharmaceutically acceptable carrier, diluent or excipient. Accordingly, provided herein are pharmaceutical compositions comprising the recombinant glycosylated protein composition (e.g., the antibody composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

The following examples are given merely to illustrate the present disclosure and not in any way to limit its scope.

### Examples

1. Introduction

To expand on the understanding of IgG2 mediated cytotoxicity, we developed highly sensitive cytotoxicity assays with panitumumab as a model IgG2 using specific responding cell types. A FcγRIIa signaling assay using an engineered cell line and reporter gene, and primary cells derived from PBMCs isolated from the whole blood of genotyped donors were deployed to study the cytotoxicity activity. We used donors expressing the common FcγRIIa and FcγRIIIa receptor allotypes. To understand the influence of quality attributes that can vary as a function of the production process, we generated panitumumab species that contained a wide range of the major glycan species, namely galactosylation, afucosylation and mannosylation and evaluated the impact to activity of each on panitumumab in various assays.

2. Materials and Methods

Panitumumab was produced in CHO cells by standard manufacturing processes at Amgen (Thousand Oaks, Calif.).
2.1 Enrichment and Enzymatic Remodeling of Glycan Species

High mannose containing species were enriched from mAbs using ProSwift ConA-1S affinity column (5×50 mm, ThermoFisher, PN 074148) on an Agilent 1100 series HPLC system with a flow rate of 0.5 mL/min. The column was first kept at initial condition with 100% buffer A (50 mM sodium acetate, 0.2M NaCl, 1 mM CaCl₂), 1 mM MgCl₂, pH 5.3) for 10.5 min, and then eluted with 100% buffer B (50 mM sodium acetate, 0.2 M NaCl, 1 mM CaCl₂), 1 mM MgCl₂. 100 mM a-methyl-mannopyranoside pH 5.3) for 17.5 min. Both flow-through and eluted fractions were collected and treated with β-(1-4)-Galactosidase (QA Bio, PN E-BG07) to remove terminal galactose. Specifically, mAb fractions were incubated with β-(1-4)-galactosidase at a ratio of 1/50 (μg/μg) in the presence of a reaction buffer containing 50 mM sodium phosphate (pH 6.0) for 1 hour at 37° C. Reactions were terminated by flash freezing.

Afucosylated species were prepared from mAbs by enzymatic treatment with Endo-H (QA-Bio, PN E-EH02). Specifically, mAbs were incubated with Endo-H for 24 hrs at 37° C. in a reaction buffer of 50 mM sodium phosphate (pH 5.5). The final mAb concentration is 4 mg/mL. Subsequently, afucosylated mAbs were separated by affinity chromatography using customized glycap-3A column (low density FcγIIIa, 3×150 mm, Zepteon, PN R3AVD1P1ML) on an Agilent 1100 series HPLC. The mobile phase A contained 20 mM Tris, 150 mM NaCl, pH 7.5 and the mobile phase B was 50 mM sodium citrate (pH 4.2). A gradient (hold at 0% B for 8 min, 0% to 18% B for 22 min) at a flow rate of 0.5 mL/min was used to separate both afucose-depleted (flow-through) and enriched (eluate) mAbs. Enzymatic treatment with β-(1-4)-galactosidase (as described above) was also carried out to remove any potential impact from terminal galactose.

Galactose remodeled samples were generated through the in vitro activity of β-1,4-galactosyltransferase (Sigma/Roche). First, fucosylated mAbs (mainly G0F) were prepared by collecting the flow-through fraction from FcγIIIa column and treated with galactosidase to remove terminal Galactose. Then, G0F enriched mAbs were incubated with β-1,4-galactosyltransferase at 37° C. in a reaction buffer containing 10 mM UDP-galactose, 100 rnM MES (pH 6.5), 20 mM MnCl₂ and 0.02% sodium azide. The final enzyme to mAb ratio is 6 (μL/rng) with a mAb concentration of 2 mg/mL. MAbs with different level of galactose were obtained by taking sample out of the reaction mixture at different time points (10 min, 20 min, 30 min, 1 hr, 2 hr, 4 hr and 9 hr) followed by flash freezing to halt the reaction.

Protein A chromatography purification was performed for all the enriched and remodeled samples to remove enzymes and other components. The purification was carried out with a prepacked protein A column (Poros A/20, 4.6×100 mm, Applied Biosystems, PN 1-5022-26) on an Agilent 1100 series HPLC system with a flow rate of 3 mL/min. After loading the appropriate amount of each sample, the column was first kept at initial condition with 100% buffer A (20 mM Tris-HCl/150 mM NaCl, pH 7.0) for 1.4 min, and then eluted with 100% buffer B (0.1% acetic acid) for 2.9 min. All eluted mAbs were diafiltered into formulation buffer using Amicon Ultra centrifugal filters with a 3 kDa cutoff membrane. Protein concentration was typically ˜1 mg/mL for all enriched/remodeled mAb samples.
2.2 Characterization of Enriched and Remodeled of Glycan Species

All the enriched and remodeled samples were characterized with Hydrophilic Interaction Liquid Chromatography (HILIC) and Size Exclusion Chromatography to ensure desired glycan properties and minimal level of high molecular weight species. Glycans from mAbs were released using PNGase F (New England BioLabs) with an E/S ratio of 1/25 (μL/μg) and labeled with 12 mg/mL 2-aminobenzoic acid (2-AA, Sigma-Aldrich) by incubating the reaction mixture at 80° C. for 75 min. 2-AA labeled glycans were separated with BEH glycan column (1.7 μm, 2.1×100 mm, Waters) on a Waters Acuity or H-Class UPLC system equipped with a fluorescence detector. The column temperature was maintained at 55° C. The mobile phase A contained 100 mM ammonium format (pH3.0) and the mobile phase B was 100% acetonitrile. Glycans were bound to the column in high organic solvent then eluted with an increasing gradients of aqueous ammonium formate buffer (76% B was held for 5 min, followed by a gradient from 76 to 65.5% B over 14 min). Confirmation that the required manipulations didn't result in the formation of high molecular weight species was assessed using a size exclusion column (SEC) TSK-Gel G3000SWLXL (7.8×300 mm, Tosoh Bioscience) on an Agilent 1100 HPLC system with a flow rate of 0.5 mL/min. Sample loads of 20-40 μg of sample were typically separated isocratically with a mobile phase containing 100 mM sodium phosphate (pH 6.8) and 250 mM NaCl.
2.3 FcγRIIa Reporter Gene Assay

The FcγRIIa reporter luciferase reporter gene assay employs engineered Jurkat T cells as the effector cells. The Jurkat reporter cells express IgG Fc receptor FcγRIIa (H131 variant) on the cell surface as well as a luciferase reporter gene with a response element for the nuclear factor of activated T cell (NFAT). Concurrent binding between an antibody on target cells and of the antibody Fc domain with stably expressed FcγRIIa on Jurkat effector cells activates the transcription factor NFAT. Activated NFAT translocates into the nucleus of Jurkat cells and induces luciferase reporter gene expression. After addition of a luciferase substrate that contains luciferin and surfactant, a luminescence signal generation enables detection of FcγRIIa reporter activity. For panitumumab, the reference standard, assay control, and test samples are serially diluted over 8 concentration levels in RPMI 1640 assay medium with low IgG FBS to the range of (0.004 μg/mL-2 μg/mL) of the final plate well concentration to serve as a dose response curve. Effector Jurkat reporter and target (A431) cells are prepared in a combined cell suspension at an effector to target (E to T) cell ratio of 3:2. The plate is then incubated in a humidified incubator at 5% CO₂ and 37° C. for about 5.5 hours. At the end of the incubation, cells are lysed by the surfactant in the luciferase assay buffer. Luminescence signal generated by the luciferase reaction with its substrate luciferin in the luciferase assay buffer is detected by an EnVision plate reader. Data were fitted to the mean emission values using a 4 parameter fit using SoftMaxPro and reported as percent activity as calculation by EC50 standard/EC50 sample. Each sample is tested in 3 independent assays and the sample final result is reported as the mean of the 3 determinations.
2.4 Donor Allotyping and Cell Cytotoxicity Assays Using PBMCs

Allotyping PBMC Donors. PBMC's were isolated from the blood of healthy donors using Becton Dickinson Cell Preparation Tubes (BD-CPT). 8 mL of blood was collected from each donor using veni-puncture into BD CPT tubes. The tubes were then centrifuged for 30 mins at 1500 RPM to separate the blood into different layers. The plasma layer was aspirated, and the lymphocytes were collected into a 15 mL centrifuge tube. The lymphocytes were then washed 2× with PBS to remove any plasma and the cells are counted before DNA isolation. DNA was extracted from cells using QIAGEN Blood and Cell Culture DNA Kit. DNA was then subjected to Taqman single nucleotide polymorphism (SNP) genotyping analysis with a qualified set specific for each receptor (FcγRIIa and FcγRIIIa) in a 7900 HT RealTime PCR system. qPCR assay was set up with master mix, DNA, and assay Oligo mix (fluorescently labeled probes) for 40 cycles. Each probe anneals specifically to a complementary sequence if present. The exonuclease activity of the DNA polymerase cleaves probes that have been hybridized to the target, releasing the reporter dye, resulting in increased fluorescence. If the specific sequence doesn't exist the probe doesn't attach during amplification, and thereby no dye will be released, so the presence of the dye is indicative of a particular polymorphism. SDS software gives a read-out of each well, and the call determines the genotype. The software also provides an allelic discrimination plot, where clustering is indicative of individual genotypes. TaqMan® 5′-nuclease assay chemistry provided a way to get single nucleotide polymorphism (SNP) genotyping results. Each predesigned TaqMan® SNP Genotyping Assay included two allele-specific TaqMan® MGB probes containing distinct fluorescent dyes and a PCR primer pair to detect specific SNP targets. These TaqMan® probe and primer sets (assays) uniquely align with the genome to provide unmatched specificity for the allele of interest. The SNP assay for FcγRIIA 131 histidine or arginine polymorphism (H/R) is C_9077561_20. The SNP assay for FcγRIIIA 158 phenylalanine or valine (F/V) is C_25815666_10.

KILR™ Cell Cytotoxicity Assay. This assay utilized U2OS target cells that overexpress EGFR and a proprietary housekeeping protein fused to an inactive fragment of the β-galactosidase (β-gal) reporter that is a component of the Eurofins DiscoverX KILR™ Cytotoxicity Assay. Modified target cells were mixed with PBMC at a 1:200 ratio, respectively, in the presence of varying concentrations of panitumumab or glycoengineered samples. When target cells were lysed the tagged housekeeping protein was released into the media. The tagged housekeeping protein is detected in the media by addition of reagents containing another fragment of the β-gal reporter which leads to the formation of an active β-gal enzyme. Upon β-gal-dependent hydrolysis of a chemiluminescent reagent, a dose dependent increase in luminescence occurs. Luminescence response data was directly proportional to the amount of cytotoxicity. Fresh PBMC from healthy donors was used in this assay as effector cells. The luminescence signal was detected with a plate reader. The luminescence response was plotted relative to the test concentration and dose response curves generated.

PBMC isolated from healthy volunteers with known FcγRIIa and FcγRIIIa genotypes were procured by Amgen (Thousand Oaks, Calif.). KILR™ ADCC assays were performed by isolating PBMC using BD-CPT tubes. PBMC were harvested, washed in D-PBS, and 1.2×10⁶ cells were dispensed per well of a 96-well plate. KILR™ U2OS target cells (6,000/well) were added to the wells containing PBMC and were incubated with increasing concentrations of panitumumab or glycoengineered samples (0.148-200 ng/mL) for 12 hours. The dose dependent increase in luminescence signal is detected by reading the assay plates on a Perkin Elmer Envision plate reader. Data analysis was performed using SoftMax Pro v5.4.1 and dose response curves are reported.
2.5 FcγR Blocking Assay to Show Specificity

Receptor Antibody blocking studies were performed by individually blocking CD16 (FcγRIIIa), CD32 (FcγRIIa) and CD64 (FcγRI) with antibodies that specifically bind and block these receptors and the resulting cytotoxic activity was measured. Panitumumab was used at a constant concentration of 200 mg/mL and varying concentrations (2000 ng/mL-1 ng/mL) of the different blocking mAbs (anti FcγRI [mouse monoclonal, BioLegend cat#360701], anti-FcγRIIa [goat polyclonal, R&D Systems cat #AF1330] and anti FcγRIIIa [goat polyclonal, R&D Systems cat #AF1257]). Goat Isotype control: polyclonal goat, R&D Systems cat #AB-108-C; Mouse IgG1/k isotype control: mouseIgG1/k, BD Biosciences cat#550979.

U2OS target cells engineered with the KILR® housekeeping gene from Eurofins DiscoverX were harvested and plated in a 96 well plate at a density of 6000 cells/well. A constant concentration of panitumumab (200 ng/mL) was mixed with blocking reagent over a range of concentrations from 2000 ng/mL to 1 ng/mL and added to the target cells. Healthy donor PBMC were used as effector cells by taking whole blood and isolating PBMCs with BD-CPT tubes. These PBMCs were then added to the mixture of target cells and antibody mixtures at a density of 1.2e6 cells/well for an effector to target ratio of 200:1. Assay plates were co-cultured at 37° C. for approximately 18 hours before the addition of KILR® Detection reagent and reading of the luminescent signal on an Envision plate reader.
2.6 FcγR Binding by SPR

Surface plasmon resonance experiments were performed using the SPR T-200 instrument. His-tagged human FcγRs were expressed in CHO cells and purified in house mouse anti-his capture antibody was immobilized at approximately 5000 RU on a Series S Sensor Chip CM5 (GE Healthcare) using Instrument Buffer (0.005% P20 in PBS). FcγRs were diluted to 3.3˜10 nM in running buffer (0.005% P20, 0.1 mg/mL BSA in PBS) and injected at 10 μL/min for 1.5 min for the capture step. Panitumumab samples were diluted in running buffer (PBS+0.005% P20+0.1 mg/mL BSA) over a range of concentrations from 0.4 nM-20000 nM and injected over the captured FcγR with association and dissociation times of 3 minutes at 50 μL/min. The chip surface was regenerated by injecting 10 mM glycine, pH 1.7 at 30 μL/min for 30 s.

3. Results
3.1 Influence of Glycan Species on Panitumumab Mediated Cytotoxicity

As has been previous described, panitumumab can mediate a cell mediated cytotoxicity activity that had not been previously described for human IgG2s therapeutic antibodies. To ascertain which product quality attributes can influence that activity, a series of enrichment and enzymatic treatments (see materials and methods) were used to alter the glycan profile of panitumumab to produce a wide range of each of the major glycan species: terminal galactose, core fucose, and high mannose. As this activity has previously been attributed to FcγRIIa, we also devised a sensitive FcγRIIa reporter gene assay to read out the impact of quality attributes on the activity.

The first glycan species evaluated for an impact was terminal β-galactose. Panitumumab samples were enzymatically treated as described in the methods section to display a wide range of terminal galactose from 0.4% to 88.3%. As shown in FIGS. 2A-2B, over that range of galactose levels, the activity as measured by the reporter gene assay ranged nearly 60%, with activity levels showing a very linear response to the galactose level. We quantified the relative impact of β-galactose on FcγRIIa signaling activity by expressing it in terms of the slope of the activity/attribute correlation plot, which can be taken to represent a response coefficient. Using this approach for panitumumab, the FcγRIIa signaling impact can be calculated as 0.6681 for β-galactose, with an R² value of 0.98.

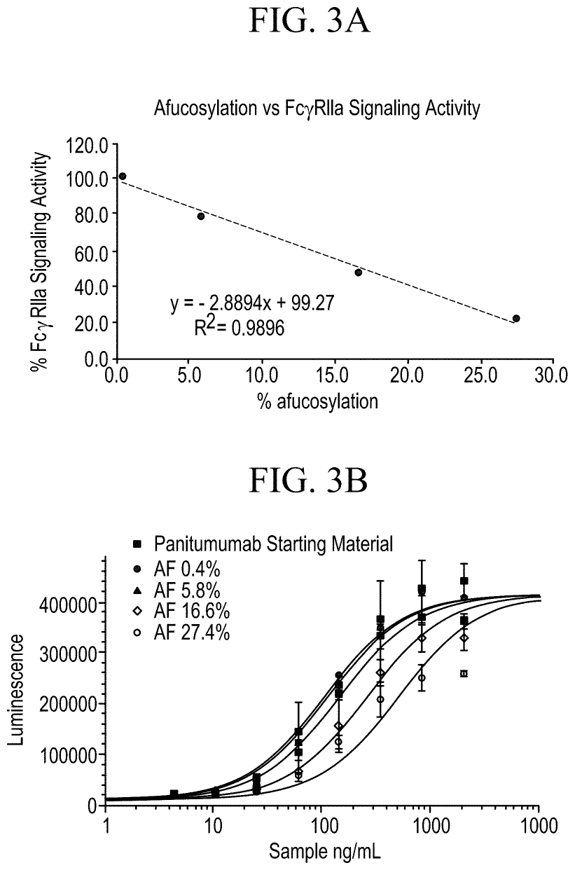

Next, we examined the influence of the level of the core fucose species on the FcγRIIa reporter gene assay. Panitumumab also demonstrated a linear response to varying fucose (afucosylation) levies. The dose response curve for FcγRIIa signaling activity for afucosylation is shown in FIGS. 3A-3B. The calculated activity yielded a very linear negative response to the amount of afucosylation. The data shows that the panitumumab mediates higher cytotoxicity at lower afucosylation levels and lower cytotoxicity at higher afucosylation levels thereby inversely correlating with percent afucosylation on the mAb. We note that this is an interesting reversal of the impact that afucose levels have on FcγRIIIa mediated ADCC activity by IgG1.

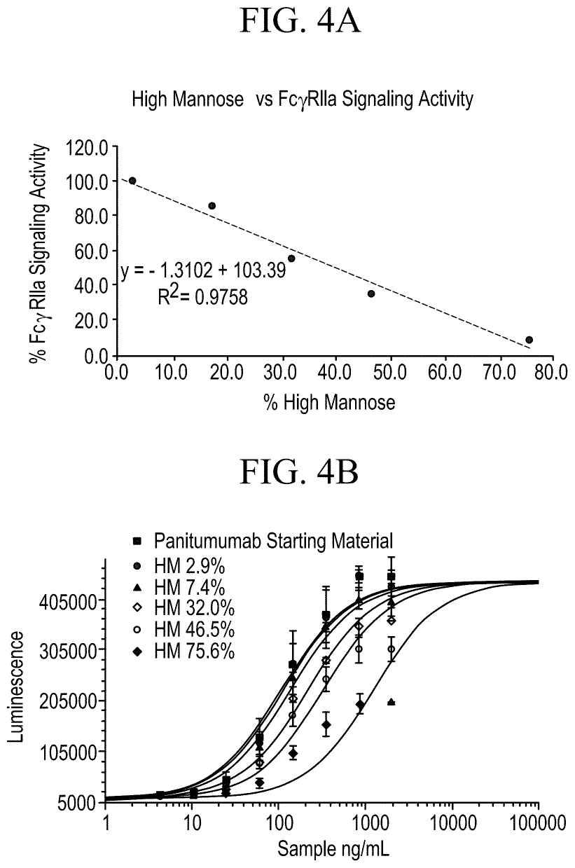

The last component of this glycan study involved examining the effect of high mannose on the ability of panitumumab to impact the FcγRIIa reporter gene assay. The FcγRIIa signaling activity response as a function high mannose level is shown in FIGS. 4A-4B. Here again panitumumab has a linear but inverse response to high mannose.

A summary of the impacts of the different glycan species can be found in Table 3.

TABLE 3
Summary of the impact of the various glycan attributes on panitumumab activity

| | Afucose | | High Mannose | | β−Galactose | |
|---|---|---|---|---|---|---|
| | Slope | | Slope | | Slope | |
| Responding Cell Type | Value | R² | Value | R² | Value | R² |
| FcγRIIa 131H Signaling | −2.88 | 0.9896 | −1.31 | 0.9758 | 0.66 | 0.9698 |
| activity | | | | | | |
| HHVV PBMC | NV | NV | −0.45 | 0.7944 | NV | 0.4647 |
| HHFF PBMC | −0.723 | 0.8958 | −1.35 | 0.7999 | ND | |
| RRFV PBMC | −0.448 | 1.000* | −0.194 | 1.000* | NV | 0.6632 |
| HHFV PBMC | −0.538 | 0.7652 | −0.178 | 0.9076 | NV | 0.0013 |
| RRFF PBMC | | | | | NV | 0.0715 |

*Data from two dose points only
NV: No value due to very low R² values
NA: None Apparent
ND: Not Done

3.2 PBMC Allotyping

In order to extend these observations to an additional assay format more reflective of a physiological context, we developed a primary PBMC assay to assess the impact of panitumumab mediated cytotoxicity, see materials and methods. And to assess the FcγR allotype impact in this method, we genotyped DNA of several donors to determine the allele at amino acid position 131 (H/R) of the FcγRIIA and the allele at amino acid position 158 (V/F) of the FcγRIIIA receptor. The allelic clusters for FcγRIIIa receptor polymorphism were 52% homozygous for FF genotype, 36% heterozygous for FV and 12% homozygous for VV at amino acid position 158 and the allelic discrimination plot for FcγRIIa found 26% homozygous for RR genotype, 58% heterozygous for HR and 16% homozygous for HH at amino acid position 131. Primary PBMCs from these donors were used in subsequent cytotoxicity activity assays.
3.3 PBMC Cytotoxicity Data

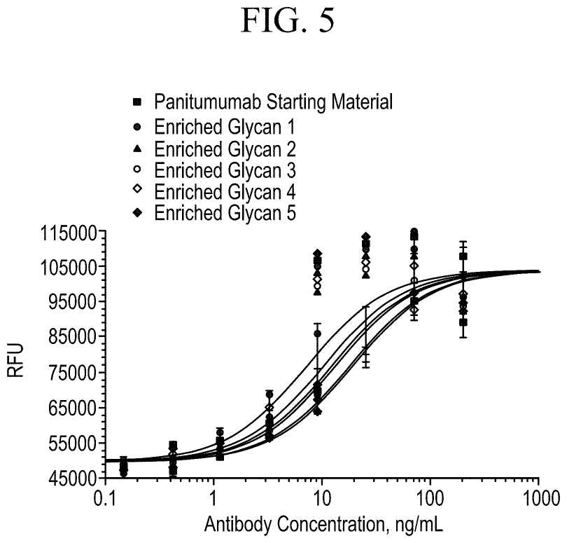

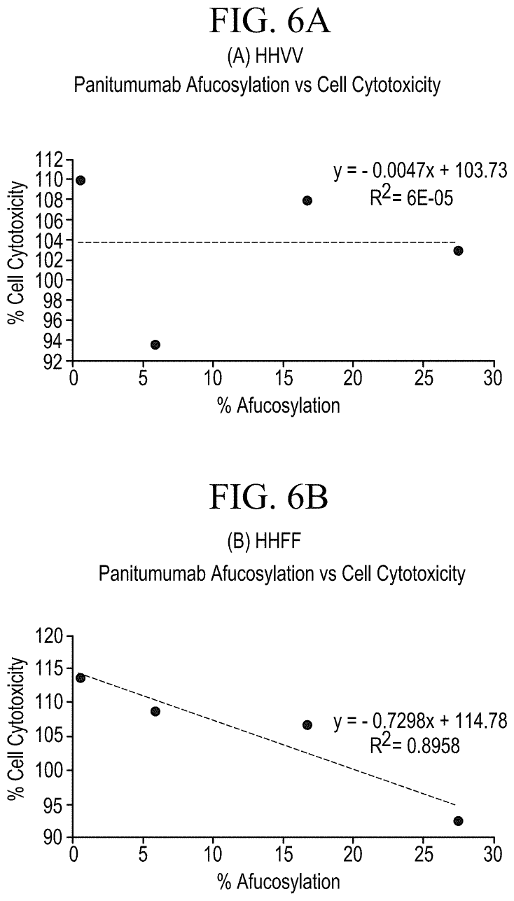

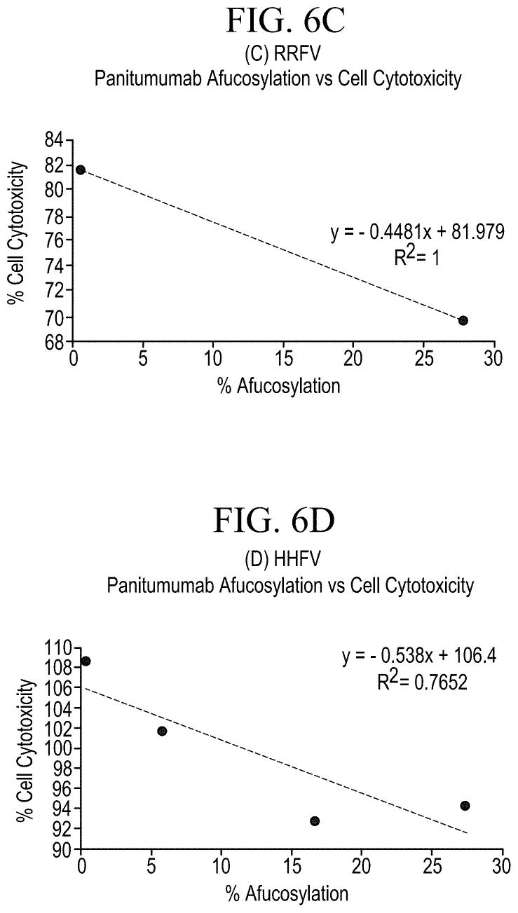

PBMC's from donors with HHVV, HHFF, RRFV and HHFV allotypes for FcγRIIa and FcγRIIIa respectively were used to test the wide ranging afucosylated, mannosylated and galactosylated Panitumumab samples in cytotoxicity assays as described in materials and methods. A representative dose-response curve overlay for the method with varying levels of a particular glycan is shown in FIG. 5. Panitumumab again showed a linear inverse relationship response to a range of fucose (afucosylation) from 0.4%-27.4%. The calculated activity yielded a very linear negative response to the amount of afucosyltion (FIGS. 6A-6D) when tested with donors with different allotypes (HHVV, HHFF, RRFV and HHFV respectively). All donors with the exception of HHVV showed a linear negative correlation of cell killing to the percent afucosylation.

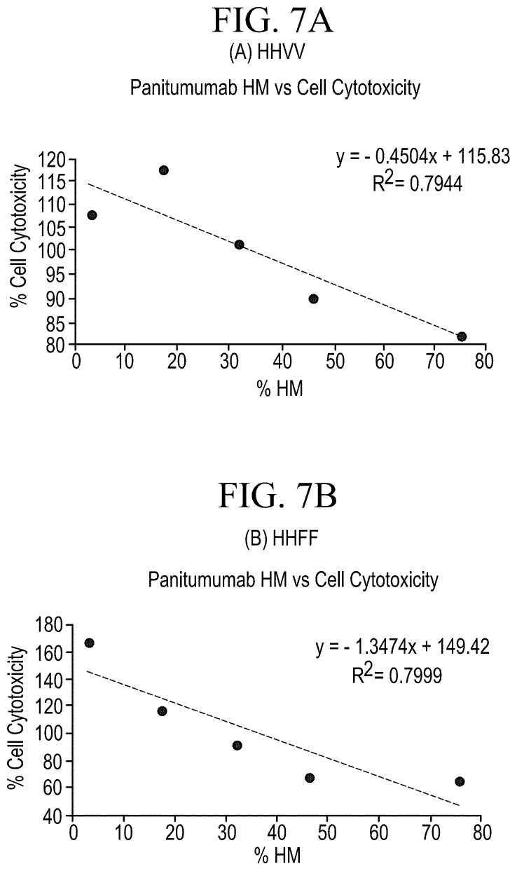

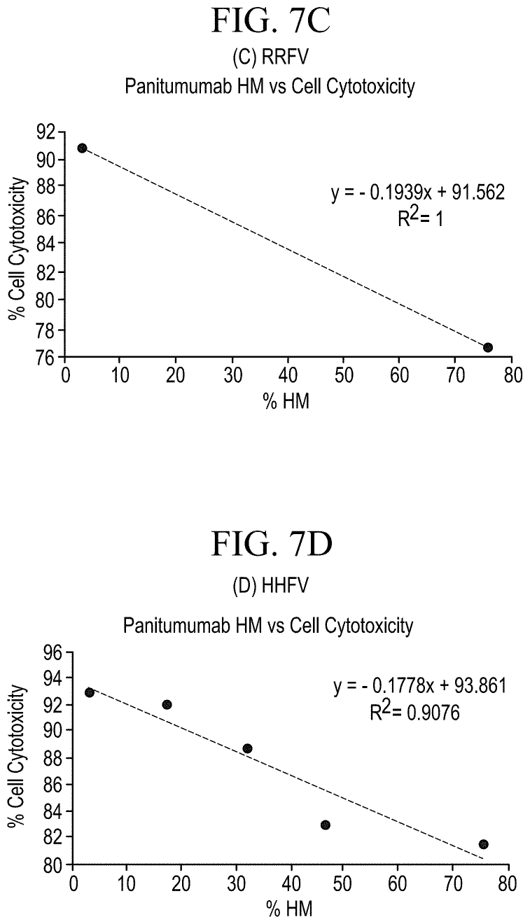

Panitumumab also again showed a linear response to a range of high mannose from 2.9%-75.6%. The calculated activity yielded a very linear negative response to the amount of high mannose (FIGS. 7A-7D) when tested with donors with different allotypes (HHVV, HHFF, RRFV and HHFV respectively). All 4 donors showed a strong negative correlation of cell killing to high mannose levels in this assay.

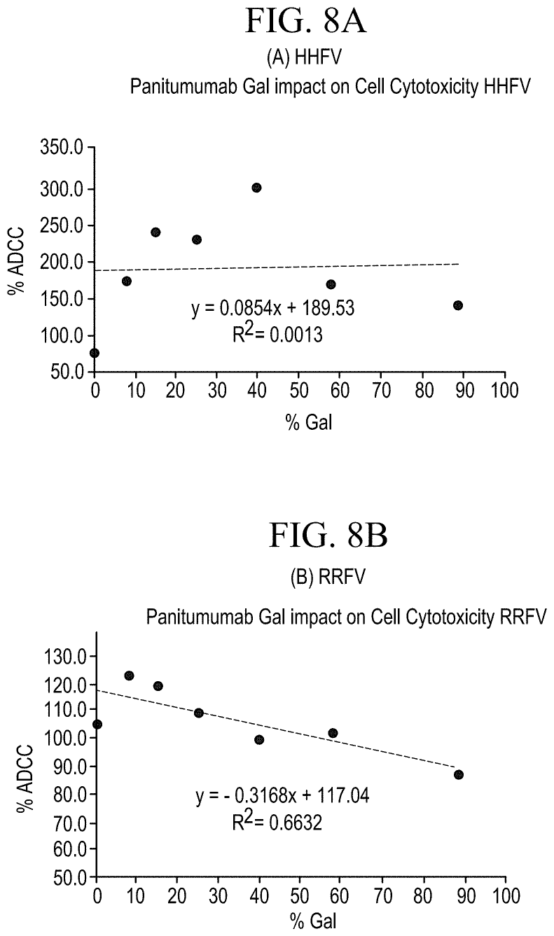

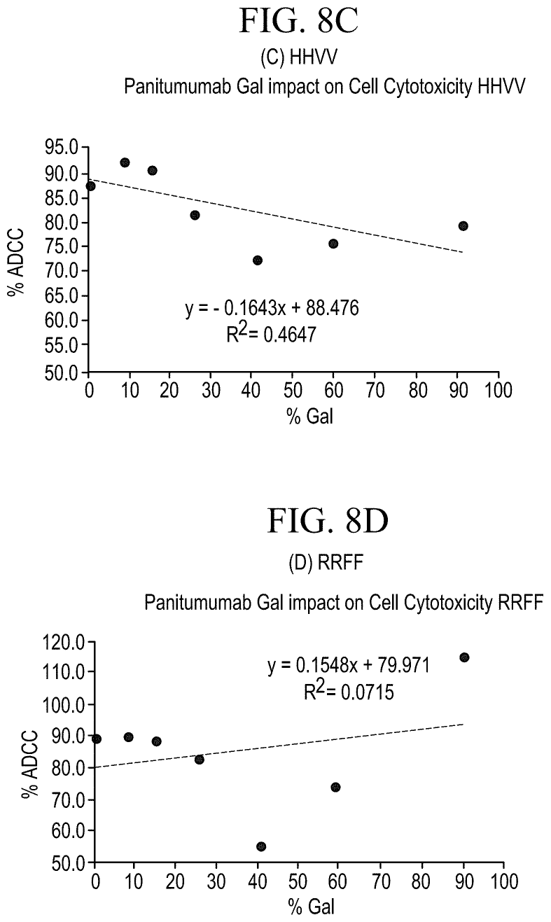

Panitumumab samples possessing a wide range of terminal galactose from 0.4% to 88.3% were also tested in PBMC mediated cytotoxicity assays. In this case, the results showed a lack of a substantial correlation between cytotoxicity activity with the varying levels of galactosylation, unlike what had been seen with the FcγRIIa reporter gene assay. We were unable to quantify the relative impact of β-galactose on cell cytotoxicity using this approach for panitumumab. The assays were done with PBMC's from 4 donors with different allotypes as shown in FIGS. 8A-8D with HHFV, RRFV, HHVV and RRFF, respectively.
3.4 FcγRIIa is the Only Receptor Involved in IgG2 Mediated Cell Killing

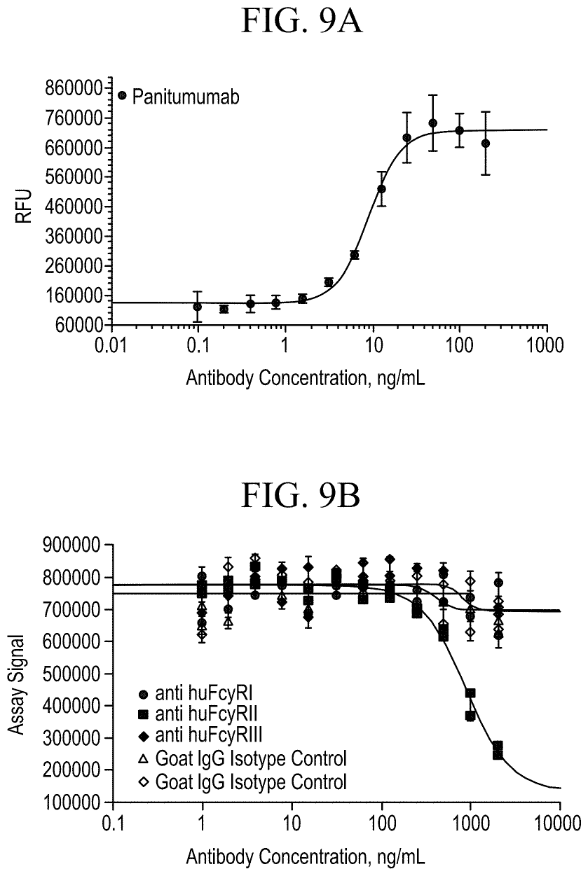

Receptor antibody blocking studies were performed by individually blocking CD16 (FcγRIIIa), CD32 (FcγRIIa) and CD64 (FcγRI) with antibodies that specifically bind and block these receptors and the resulting cytotoxic activity was measured. Blocking experiment was set up using the KILR assay with PBMC's and Panitumumab. Panitumumab was used at a constant concentration of 200 mg/mL and varying concentrations (2000 ng/mL-1 ng/mL) of the different blocking mAbs showed that only cells incubated with anti-FcγRIIa showed reduction in cell death with increasing concentrations of the blocking mAb. Cells treated with Panitumumab (0.1-200 ng/mL) without any blocking antibodies mediated cell cytotoxicity as expected in a dose dependent manner (FIG. 9A). The cells incubated with anti FcγRI or anti FcγRIIIa showed no difference in percent cell death at any concentration of the blocking mAb similar to isotype control mAbs (FIG. 9B). This demonstrates that the IgG2 (Panitumumab) mediated cytotoxicity is mainly through the involvement of FcγRIIa receptor and not FcγRI or FcγRIIIa.
3.5 Panitumumab Binding to FcγRs

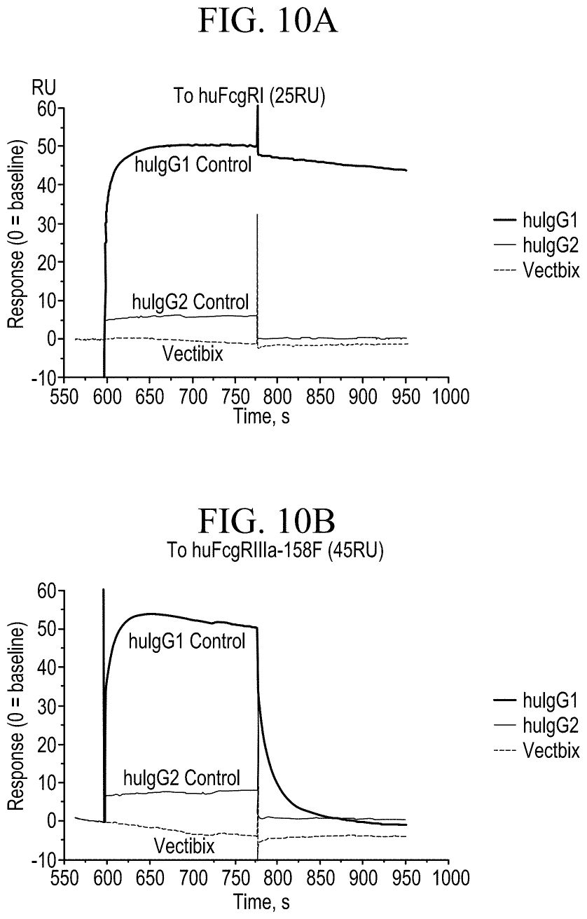

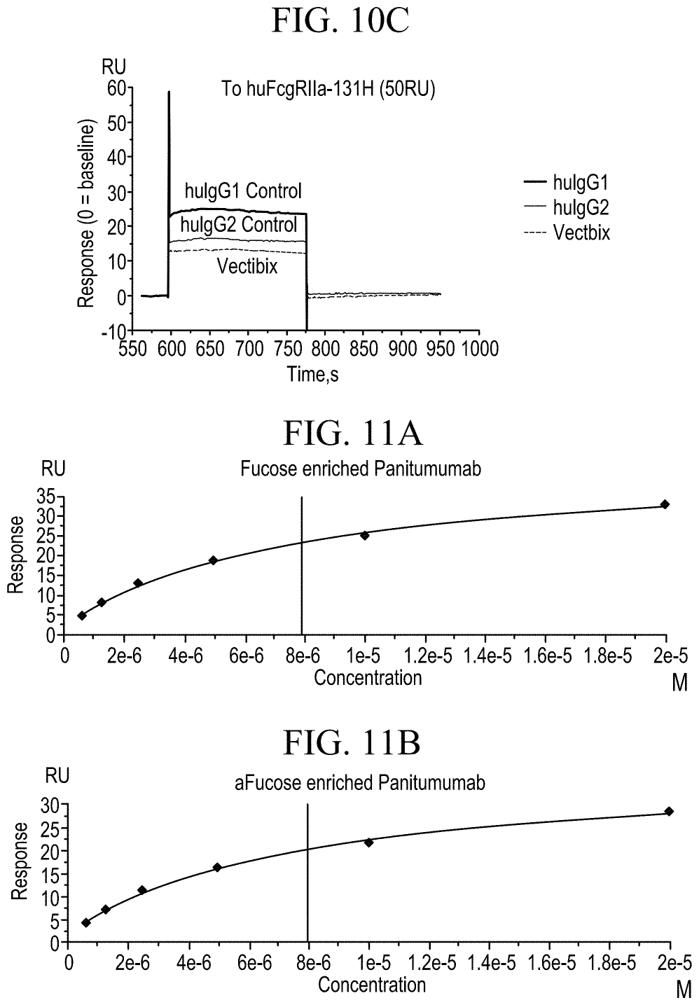

To try to assess the affinity of panitumumab and the various enriched glycan species for FcγRs, we measured the biding of panitumumab samples to all three human Fcγ Receptors by surface plasmon resonance (SPR). Panitumumab at 10 μM did not show any detectable binding to human FcγRI or FcγRIIIa-158F but did bind to huFcγRIIa-131H (FIG. 10A-10C). Panitumumab and hulgG2 control bound to huFcγRIIa-131H with apparent KD˜20 μM and 25 μM, respectively (FIG. 10C). The binding was further assessed for the fucose and afucose enriched panitumumab samples by equilibrium binding (FIGS. 11A-11B). The difference in binding by SPR between the two glycan enriched samples (fucose enriched and afucose enriched K_{D} values of ˜7.9 μM and 8 μM respectively) was not nearly as pronounced as the differences found in the FcγRIIa signaling activity or PBMC cytotoxicity assays. This is likely attributable to the signal amplification afforded by cell-based assays through the receptor clustering, signal transduction and gene expression aspects of the binding response (see, e.g., Unkeless et al, Semin Immunol. 1995; 7(1):37-44; Amigorena et al., Science. 1992; 256(5065):1808-1812; Amigorena et al., Nature. 1992; 358(6384):337-341; Regnault et al., J Exp Med. 1999; 189(2):371-380).

To summarize the impact of the various glycans on panitumumab cytotoxic activity, Table 3 shows the slope values and R² for each donor when tested with either afucosylated samples, high mannose samples or β-galactose samples for their ability to mediate cell cytotoxicity.

4. Discussion

The goal of this study to understand the mechanism and product quality attributes that affect therapeutic human IgG2 monoclonal antibody mediated cytotoxicity. To assess the impact of quality attributes, it is necessary to have highly sensitive, reproducible, quantitative functional assays. To understand the influence of quality attributes that can vary as a function of the production process, we generated panitumumab species that contained a wide range of the major glycan species, namely galactosylation, afucosylation and mannosylation and evaluated the impact to activity of each on panitumumab in various assays. Through the engineering process we were able to achieve ranges substantially wider than would be possible by process modifications to more accurately discern the relationship between attribute and activity. Gal levels ranged from 0.4% to 88.3%, afucose levels ranged from 0.4% to 27.4% and high mannose levels ranged from 2.9% to 75.6%.

Many assays using primary cells to determine phagocytic activity is prone to inconsistency in effector type present in the population derived from the donor at the time of the assay, as well as receptor allotypes and other background genetic variability that could affect the assay activity. Also, it is generally more difficult to discern the relevant receptors on phagocytes due to the diversity of receptors expressed as others have also noted (Parren et al., J. Clin. Invest. 90: 1537-1546, 1992; Salmon et al, 1992, J. Clin. Invest. 89(4):1274-81; Ackerman et al., J Immunol Methods. 2011; 366:8-19). From an operational perspective, assay throughput is also limited by the number of cells that can be harvested. Consequently, these types of assays are ill-suited to drug development and characterization in a quality-control setting. In recognizing of these challenges, Tada et el (PLOS ONE, 2 Apr. 2014, Volume 9, Issue 4, e95787) developed a reporter gene assay for FcγRIIa signaling activity which overcomes many of the above limitations.

Effector functions are also dependent on receptor polymorphisms (158V or F for FcγRIIIa or 131H or R for FcγRIIa). To further study the effect of quality attributes in a more physiologically relevant setting, we used PBMC donors expressing the common FcγRIIa and FcγRIIIa receptor allotypes to assess if there is any impact on receptor allotype on the panitumumab mediated cytotoxicity and conclusions about glycan impact. We were able to develop a reliable functional assay using PBMC's that was able to tolerate long read out essays as the kinetics of IgG2 is much slower than IgG1 thereby increasing the duration of incubation for these assays. KILR (killing immuno lysis reaction) by DisCoverX is a non-radioactive assay for kinetically slow cytotoxicity. KILR assays were set up using panitumumab coated U2OS target cells overexpressing EGFR transduced with KILR Housekeeping gene and PBMC's as effector cells.

These studies demonstrate for the first time that the glycans in IgG2 panitumumab have substantial and differential impact on cell cytotoxicity activity. We observed afucosylation and mannosylation have a negative impact on cell cytotoxicity both in the FcγRIIa signaling activity and in PBMC mediated cell killing assays, a phenomenon completely opposite to IgG1. Increase in afucosylation significantly reduces cell killing ability of the antibody and similarly increase in high mannose content in the antibody decreases cell cytotoxicity mediated by panitumumab. A very linear negative response is observed in both the primary cell and reporter gene assay for these two glycans. Galactosylation on the other hand seems to have a more modest but positive correlation between β-galactose content and cell killing. This was more pronounced in the FcγRIIa signaling activity assays than in PBMC mediated cell killing assays. It should be mentioned that there is as yet no definitive mechanistic explanation available for the observed association of FcγRIIa to IgG2, because the affinity of fucosylated panitumumab to FcγRIIa-H131 (KD˜7.9 μM) appears only slightly higher than that for afucosylated panitumumab to FcγRIIa-H131 (KD˜8 μM), as measured by surface plasmon resonance.

It is acknowledged that PMBCs are a complex and variable population. To confirm that FcγRIIa was mediating the activity, receptor blocking studies were performed to confirm the specificity to show Panitumumab mediates cytotoxicity through FcγRIIa and not FcγRIIIa. This was accomplished by using blocking antibodies against FcγRI, FcγRIIa and FcγRIIIa. Cytotoxicity was inhibited only with increasing concentrations of anti-FcγRIIa and had no impact on cytotoxicity when blocked with the other antibodies against FcγRI or FcγRIIIa. The difference in cytotoxicity levels between the different donors with the same FcγRIIa alleles could be due to various reasons, such as receptor density, membrane mobility or interactions/cooperation with other molecules that could potentially affect intracellular signaling and consequently cell cytotoxicity. It remains to be discerned if and how the role of inhibitory receptors affects the overall cell cytotoxicity in PBMCs.

The FcγR involvement was further examined by SPR binding assays where panitumumab samples upto 10 μM was tested for binding to FcγRI, FcγRIIa 131H and FcγRIIIa 158V. Binding was detected only to FcγRIIa 131H with an apparent KD of 20 μM. IgG2 control mAb used in this study also demonstrated similar binding activity to only FcγRIIa at 25 μM KD. Additionally, fucose enriched and afucose enriched samples were generated to detect differences in binding activity. In the context of the SPR binding assay, a significant difference in binding between the two glycan enriched samples and the fucose and afucose enriched was not as dramatic as was seen in the functional assays, with samples binding with 7.9 μM and 8 μM KD respectively. Engagement of immunoreceptor tyrosine-based activation motif (ITAM)-bearing type FcγRs by IgG complexes initiates a number of signaling cascades that lead to cellular activation and subsequent induction of effector functions. Cellular responses to Fc-FcγR interactions vary between myeloid cell types; however, FcγR aggregation typically leads to rapid internalization of FcγRs and activation of different signaling pathways that influence cell activation (Unkekess et al., Semin Immunol. 1995; 7(1):37-44. PubMed PMID: 7612894; Amigorena et al., Science. 1992; 256(5065)1808-1812; Amigorena et al., Nature. 1992; 358(6384):337-341; Regnault et al., J Exp Med. 1999; 189(2):371-380). The differences between the afucose and fucosylated Panitumumab might be less obvious in a Biacore binding assay due to these missing components like clustering and signal amplification that a cell-based assay might bring about.

In this study, the wide-ranging attributes combined with highly responsive functional assays revealed a significant impact to a novel IgG2 mediated cytotoxicity activity by the conserved Fc glycan. This understanding should be taken into consideration during the design and characterization of therapeutic IgG2 drug candidates.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the disclosed embodiments. The section headings used herein are for organizational purposes only and are not to be construed as limiting the subject matter described.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The present invention relates in particular to the following embodiments:

1. A method of modulating Fc gamma Receptor (FcγR)-mediated cytotoxicity of panitumumab, comprising increasing or decreasing the amount of terminal β-galactose at the N-297 glycosylation site of panitumumab, or increasing or decreasing the amount of panitumumab molecules that comprise G1, G1a, G1b and/or G2 galactosylated glycan at the N-297 site.
2. The method of claim 1, wherein the FcγR-mediated cytotoxicity of panitumumab is increased by increasing the amount of terminal β-galactose at the N-297 glycosylation site of panitumumab, or increasing the amount of panitumumab molecules that comprise G1, G1a, G1b and/or G2 galactosylated glycan at the N-297 site.
3. The method of claim 1, wherein the FcγR-mediated cytotoxicity of panitumumab is decreased by decreasing the amount of terminal β-galactose at the N-297 glycosylation site of panitumumab, or increasing the amount of panitumumab molecules that comprise G1, G1a, G1b and/or G2 galactosylated glycan at the N-297 site.
4. The method of claim 1, wherein said FcγR is FcγRIIa.
5. The method of claim 1, wherein said FcγR-mediated cytotoxicity is FcγRIIa-mediated cellular cytotoxicity.
6. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of terminal β-galactose at the N-297 glycosylation site of panitumumab, or increasing or decreasing the amount of panitumumab molecules that comprise G1, G1a, G1b and/or G2 galactosylated glycan at the N-297 site; such that the difference in FcγR-mediated cytotoxicity between the panitumumab sample and the reference value is about 35% or less.
7. The method of claim 6, wherein the FcγR-mediated cytotoxicity of the panitumumab sample is increased by increasing the amount of terminal β-galactose at the N-297 glycosylation site of panitumumab, or increasing the amount of panitumumab molecules that comprise G1, G1a, G1b and/or G2 galactosylated glycan at the N-297 site.
8. The method of claim 6, wherein the FcγR-mediated cytotoxicity of the panitumumab sample is decreased by decreasing the amount of terminal β-galactose at the N-297 glycosylation site of panitumumab, or decreasing the amount of panitumumab molecules that comprise G1, G1a, G1b and/or G2 galactosylated glycan at the N-297 site.
9. The method of claim 6, wherein said FcγR is FcγRIIa.
10. The method of claim 6, wherein said FcγR-mediated cytotoxicity is FcγRIIa-mediated cellular cytotoxicity.
11. A method of modulating Fc gamma Receptor (FcγR)-mediated cytotoxicity of panitumumab, comprising increasing or decreasing the amount of panitumumab molecules that comprise fucosylated glycan at the N-297 site, or increasing or decreasing the amount of panitumumab molecules that comprise afucosylated glycan at the N-297 site.
12. The method of claim 11, wherein the FcγR-mediated cytotoxicity of panitumumab is increased by increasing the amount of panitumumab molecules that comprise fucosylated glycan at the N-297 site, or decreasing the amount of panitumumab molecules that comprise afucosylated glycan at the N-297 site.
13. The method of claim 11, wherein the FcγR-mediated cytotoxicity of panitumumab is decreased by decreasing the amount of panitumumab molecules that comprise fucosylated glycan at the N-297 site, or increasing the amount of panitumumab molecules that comprise afucosylated glycan at the N-297 site.
14. The method of claim 11, wherein said FcγR is FcγRIIa.
15. The method of claim 11, wherein said FcγR-mediated cytotoxicity is FcγRIIa-mediated cellular cytotoxicity.
16. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise fucosylated glycan at the N-297 site, or by increasing or decreasing the amount of panitumumab molecules that comprise afucosylated glycan at the N-297 site; such that the difference in FcγR-mediated cytotoxicity between the panitumumab sample and the reference value is about 35% or less.
17. The method of claim 16, wherein the FcγR-mediated cytotoxicity of the panitumumab sample is increased by increasing the amount of panitumumab molecules that comprise fucosylated glycan at the N-297 site, or decreasing the amount of panitumumab molecules that comprise afucosylated glycan at the N-297 site.
18. The method of claim 16, wherein the FcγR-mediated cytotoxicity of the panitumumab sample is decreased by decreasing the amount of panitumumab molecules that comprise fucosylated glycan at the N-297 site, or increasing the amount of panitumumab molecules that comprise afucosylated glycan at the N-297 site.
19. The method of claim 16, wherein said FcγR is FcγRIIa.
20. The method of claim 16, wherein said FcγR-mediated cytotoxicity is FcγRIIa-mediated cellular cytotoxicity.
21. A method of modulating Fc gamma Receptor (FcγR)-mediated cytotoxicity of panitumumab, comprising increasing or decreasing the amount of panitumumab molecules that comprise a high-mannose glycan at the N-297 site.
22. The method of claim 21, wherein the FcγR-mediated cytotoxicity of panitumumab is increased by decreasing the amount of panitumumab molecules that comprise a high-mannose glycan at the N-297 site.
23. The method of claim 21, wherein the FcγR-mediated cytotoxicity of panitumumab is decreased by increasing the amount of panitumumab molecules that comprise a high-mannose glycan at the N-297 site.
24. The method of claim 21, wherein said FcγR is FcγRIIa.
25. The method of claim 21, wherein said FcγR-mediated cytotoxicity is FcγRIIa-mediated cellular cytotoxicity.
26. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise a high-mannose glycan at the N-297 site; such that the difference in FcγR-mediated cytotoxicity between the panitumumab sample and the reference value is about 35% or less.
   27. The method of claim 26, wherein the FcγR-mediated cytotoxicity of the panitumumab sample is increased by decreasing the amount of panitumumab molecules that comprise a high-mannose glycan at the N-297 site.
   28. The method of claim 26, wherein the FcγR-mediated cytotoxicity of the panitumumab sample is decreased by increasing the amount of panitumumab molecules that comprise a high-mannose glycan at the N-297 site.
   29. The method of claim 26, wherein said FcγR is FcγRIIa.
   30. The method of claim 26, wherein said FcγR-mediated cytotoxicity is FcγRIIa-mediated cellular cytotoxicity.
   31. A method of increasing Fc gamma Receptor (FcγR)-mediated cytotoxicity of panitumumab, comprising:
(i) increasing the amount of terminal β-galactose at the N-297 glycosylation site of panitumumab, or increasing the amount of panitumumab molecules that comprise G1, G1a, G1b and/or G2 galactosylated glycan at the N-297 site;
(ii) increasing the amount of panitumumab molecules that comprise fucosylated glycan at the N-297 site, or decreasing the amount of panitumumab molecules that comprise afucosylated glycan at the N-297 site; and/or
(iii) decreasing the amount of panitumumab molecules that comprise a high-mannose glycan at the N-297 site.
   32. A method of decreasing Fc Receptor (FcγR)-mediated cytotoxicity of panitumumab, comprising:
(i) decreasing the amount of terminal β-galactose at the N-297 glycosylation site of panitumumab, or decreasing the amount of panitumumab molecules that comprise G1, G1a, G1b and/or G2 galactosylated glycan at the N-297 site;
(ii) decreasing the amount of panitumumab molecules that comprise fucosylated glycan at the N-297 site, or increasing the amount of panitumumab molecules that comprise afucosylated glycan at the N-297 site; and/or
(iii) increasing the amount of panitumumab molecules that comprise a high-mannose glycan at the N-297 site.

What is claimed is:

1. A method of modulating Fc gamma Receptor (FcγR)-mediated cytotoxicity of panitumumab, comprising increasing or decreasing the amount of terminal β-galactose at the N-297 glycosylation site of panitumumab, or increasing or decreasing the amount of panitumumab molecules that comprise G1, G1a, G1b and/or G2 galactosylated glycan at the N-297 site.

2. A method of modulating Fc gamma Receptor (FcγR)-mediated cytotoxicity of panitumumab, comprising increasing or decreasing the amount of panitumumab molecules that comprise fucosylated glycan at the N-297 site, or increasing or decreasing the amount of panitumumab molecules that comprise afucosylated glycan at the N-297 site.

3. A method of modulating Fc gamma Receptor (FcγR)-mediated cytotoxicity of panitumumab, comprising increasing or decreasing the amount of panitumumab molecules that comprise a high-mannose glycan at the N-297 site.

4. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of terminal β-galactose at the N-297 glycosylation site of panitumumab, or increasing or decreasing the amount of panitumumab molecules that comprise G1, G1a, G1b and/or G2 galactosylated glycan at the N-297 site; such that the difference in FcγR-mediated cytotoxicity between the panitumumab sample and the reference value is about 35% or less.

5. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise fucosylated glycan at the N-297 site, or by increasing or decreasing the amount of panitumumab molecules that comprise afucosylated glycan at the N-297 site; such that the difference in FcγR-mediated cytotoxicity between the panitumumab sample and the reference value is about 35% or less.

6. A method of matching the Fc gamma Receptor (FcγR)-mediated cytotoxicity of a panitumumab sample to a reference value, comprising:
(1) obtaining a reference value of FcγR-mediated cytotoxicity;
(2) determining the FcγR-mediated cytotoxicity of said panitumumab sample; and
(3) changing the FcγR-mediated cytotoxicity of said panitumumab sample by increasing or decreasing the amount of panitumumab molecules that comprise a high-mannose glycan at the N-297 site; such that the difference in FcγR-mediated cytotoxicity between the panitumumab sample and the reference value is about 35% or less.

7. The method of any of claims 1 to 6, wherein the FcγR-mediated cytotoxicity of panitumumab is increased by increasing the amount of terminal β-galactose at the N-297 glycosylation site of panitumumab, or increasing the amount of panitumumab molecules that comprise G1, G1a, G1b and/or G2 galactosylated glycan at the N-297 site.

8. The method of any of claims 1 to 6, wherein the FcγR-mediated cytotoxicity of panitumumab is decreased by decreasing the amount of terminal β-galactose at the N-297 glycosylation site of panitumumab, or decreasing the amount of panitumumab molecules that comprise G1, G1a, G1b and/or G2 galactosylated glycan at the N-297 site.

9. The method of any of claims 1 to 6, wherein said FcγR is FcγRIIa.

10. The method of claim 1, wherein said FcγR-mediated cytotoxicity is FcγRIIa-mediated cellular cytotoxicity.


SEQUENCE LISTING (4 sequences)

SEQ ID NO: 1 - protein, 445 aa, Homo sapiens
  MISC_FEATURE: N-297 glycosylation site
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 214 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1416 bp, Homo sapiens
atggacctcctgtgcaagaacatgaaacacctgtggttcttcctcctcctggtggcagctcccagatgggtcctgtcccaggtacagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagtcgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcttcagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - DNA, 711 bp, Homo sapiens
atggacatgagggtccctgctcagctcctggggctcctgctgctctggctctcaggtgccagatgtgacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag